(19) United States
(12) Patent Application Publication
(10) Pub. No.: US 2026/0216057 A1
(43) Pub. Date: Jul. 30, 2026

(54) PHARMACEUTICAL COMPOSITIONS FOR HERPES VIRUS

(71) Applicant: Assembly Biosciences, Inc., South San Francisco, CA (US)

(72) Inventors: Zhixin ZONG, South San Francisco, CA (US); Nicole WHITE, South San Francisco, CA (US); Yi WU, South San Francisco, CA (US)

(73) Assignee: Assembly Biosciences, Inc., South San Francisco, CA (US)

(21) Appl. No.: 19/106,392

(22) PCT Filed: Aug. 28, 2023
(86) PCT No.: PCT/IB2023/058488

§ 371 Date: Feb. 25, 2025

Related U.S. Application Data
(60) Provisional application No. 63/373,774, filed on Aug. 29, 2022.
(60) Provisional application No. 63/484,838, filed on Feb. 14, 2023.

(51) Int. Cl. A61K 9/00; A61K 9/10; A61K 31/4439; A61K 31/513; A61K 47/18; A61K 47/20; A61K 47/22

(52) U.S. Cl. CPC A61K 9/0019; A61K 9/10; A61K 31/4439; A61K 31/513; A61K 47/18; A61K 47/20; A61K 47/22

(57) ABSTRACT

The present invention relates to methods and compositions for treating and/or inhibiting the development or progression of diseases or disorders caused by, or associated with, herpes virus infection. In particular, provided are long-acting injectable depot pharmaceutical compositions comprising a helicase-primase inhibitor; methods for their manufacture; and the use of said pharmaceutical compositions as a medicament and for the treatment of diseases or disorders caused by, or associated with, herpes virus.

### FIELD OF THE INVENTION

Provided herein are compositions and methods for treating and/or inhibiting the development or progression of diseases or disorders caused by, or associated with, herpes virus infection. In particular, provided herein are long-acting injectable depot pharmaceutical compositions comprising a helicase-primase inhibitor; methods for their manufacture; and the use of said pharmaceutical compositions as a medicament and for the treatment of diseases or disorders caused by, or associated with, herpes virus.

### BACKGROUND OF THE INVENTION

Human herpes viruses are large-enveloped double-stranded DNA viruses that share the characteristic of establishing life-long infections in humans. This is accomplished by their ability to exist in the host either as a symptom free latent infection, where the virus lies dormant or, following activation, as a lytic infection with associated symptoms. These viral infections have widespread, worldwide prevalence and it is notable that over 90% of all humans are chronically infected with more than one human herpes virus.

Human herpes viruses are classified into three subfamilies (α, β and γ) based upon their biological characteristics and the family consists of eight members, i.e., Herpes Simplex Virus subtype type 1 and 2 (HSV1, HSV2), Varicella Zoster Virus (VZV), Epstein-Barr virus (EBV), Cytomegalovirus (CMV), and Human Herpes Viruses 6-8 (HHV 6-8).

HSV1 and 2 infections can cause disease in immune competent individuals. Both subtypes cause cutaneous genital/anal and oro-labial/nasal cavity (cold sore) lesions, although HSV2 is more commonly associated with the former and HSV1 the latter. It is believed that >80% of genital infections are caused by HSV2. Globally, over 500 million people have genital herpes infections and approximately 50 to 80% of the world's population have oro-labial HSV infection, which is the main cause of cold sores. HSV, and particularly HSV1, can also cause lesions on the fingers (Whitlows) and other areas of the skin.

The vast majority of HSV infected individuals will not experience any noticeable symptoms. However, some will experience recurrent (and often severe) outbreaks of infection. In the USA, 20 to 40% of the population will get recurrent labial HSV lesions. Significantly, oro-labial cold sores and Whitlow's provide a very easy route for transmission of the virus to other individuals which can lead to rarer but much more serious HSV-related pathologies. For example, HSV-related ocular keratitis is a major cause of blindness and HSV can also cause encephalitis in neonates, which is a life-threatening condition. Other disorders believed to be caused by HSV include herpes gladiatorum, Mollaret's meningitis and possibly Bell's palsy.

Primary infection with, or reactivation of an existing herpes virus infection, can be a major cause of disease in immunocompromised individuals. Key at-risk populations include patients undergoing solid organ or stem cell transplantation, patients undergoing cancer treatment, individuals with HIV/AIDS, and ICU patients.

Presently, there is no cure for HSV. Medicines have been developed that can to some degree reduce the occurrence and/or shorten the length of outbreaks, but there is a need for improved therapies.

Currently, nucleoside analogues, such as acyclovir and its prodrugs, e.g., valacyclovir and famciclovir, are used as agents against herpes viruses such as HSV. In order to exert their effects, these nucleoside analogues must be phosphorylated by viral thymidine kinase (TK) and subsequently converted by cellular kinases to the nucleoside triphosphate, which inhibits the activity of the viral DNA polymerase. If the virus has no functionally active TK, as is the case, for example, with resistant HHV1 mutants or with TK-negative viruses, the nucleoside analogues are unable to exert their effects.

Nucleoside analogues are clinically administered at very high doses, e.g., doses as high as several hundred milligrams to several grams are typically administered per day. Even at these high doses, which are often administered over long treatment durations, these drugs are unable to completely prevent recurrent outbreaks of symptoms from HSV infection. Nucleoside analogues also do little to address the issue of viral shedding, which can asymptomatically facilitate the transmission of HSV to more individuals. Certain nucleoside analogues, particularly when used at high doses, also give rise to safety concerns. For example, since these agents can incorporate into the genome DNA of a host via the host DNA polymerase, their mutagenicity is of concern, as documented for the nucleoside analogue, ganciclovir (Aoki, Chapter 45 in Mandell, Douglas and Bennett's Principles and Practice of Infectious Diseases (Eighth Edition) 2015).

Given the inadequacy of existing treatments, there is an urgent medical need to develop improved, well-tolerated anti-herpes treatments.

One class of compounds currently being investigated are the helicase-primase inhibitors. Helicase-primase inhibitors are antiviral agents with a novel mechanism of action. They inhibit the viral heterotrimeric complex consisting of helicase, primase, and cofactor subunits, which have functions that are essential for viral DNA replication. These agents are not nucleoside analogues and do not require phosphorylation by TK to inhibit HSV replication and they are therefore potentially active against TK-deficient HSV, which as described above, is a major mechanism of resistance to nucleoside analogues.

Two examples of helicase-primase inhibitors are BILS-179 BS (Crute et al., (2002) Nature Medicine 8, p. 386-391) and amenamevir (Katsumata et al. (2018) Biochem Pharm 158 p. 201-206).

BILS-179 BS has been dosed orally to humans but was suspended in early clinical trials due to adverse events (Ruebsamen et al., (2019) Med. Chem. Commun., DOI: 10.1039/C9MD00233B). Similarly, amenamevir, which has also been dosed orally to humans, was suspended from early HSV clinical trials due to adverse events. In a dose-finding, placebo-controlled study with 437 patients with recurrent genital herpes, amenamevir was administered orally at one of four doses, namely 100 mg, 200 mg, 400 mg, and 1200 mg. It was found that the time to lesion healing, i.e., the primary endpoint of the study, was only significantly different between the very highest 1200 mg single dose group and the placebo-tested group (Aoki, Chapter 45 in Mandell, Douglas and Bennett's Principles and Practice of Infectious Diseases (Eighth Edition) 2015).

Another example of a helicase-primase inhibitor is pritelivir, a thiazolylamide derivative with the chemical name N-Methyl-N-(4-methyl-5-sulfamoyl-1,3-thiazol-2-yl)-2-[4-(pyridin-2-yl)phenyl]acetamide. The compound has been disclosed in WO 2001/47904.

In a human clinical study, the effect of pritelivir on suppression of genital herpes was studied in 156 individuals (Wald et al., (2014) New England Journal of Medicine 370, p. 201-210). Subjects received one of three oral daily doses or one weekly oral dose of pritelivir or placebo for 28 days. The four pritelivir dosing regimens were; a loading dose of 20 mg followed by a daily dose of 5 mg; a loading dose of 100 mg followed by a daily dose of 25 mg; a loading dose of 300 mg followed by a daily dose of 75 mg; and a weekly dose of 400 mg. At high doses, it was found that pritelivir reduced the rate of genital HSV shedding, the primary end point for the study. Pritelivir also reduced the number of days with lesions in otherwise healthy men and women with genital herpes. An oral daily dose of 75 mg had the greatest antiviral effect and this was found to be superior to the less frequently administered weekly dose of 400 mg.

As highlighted in the study report, although the HSV shedding rate was reduced at the highest daily dose of 75 mg, as compared with placebo, break-through shedding remained. When discussing this finding, it is explained in Wald et al., ((2014) New England Journal of Medicine 370, p. 201-210) that persistent, low-level shedding has also been observed with nucleoside therapy and that the pathogenesis of break-through viral shedding during adequate antiviral therapy with nucleoside analogues is poorly understood; it is not related to lack of adherence to the treatment regimen or viral resistance. The author also raises the question of whether further increases in the daily dose of pritelivir would completely abrogate viral shedding and should be addressed in subsequent clinical studies.

In accordance with this suggestion, a subsequent clinical study using an even higher daily oral dose was indeed conducted. In a Phase II 28-day clinical study of 91 subjects with recurrent genital HSV-2, daily oral dosing of 100 mg pritelivir (after a loading dose of 400 mg) resulted in HSV shedding in 2.4% of genital swabs analysed, compared with HSV shedding in 5.3% of swabs following daily dosing of 500 mg of valacyclovir. The results of the study are reported in Wald et al., (2016) (J. Am. Med. Assoc. 316(23), p. 2495-2503) and the publication mentions that the 100 mg pritelivir daily dose (after a loading dose of 400 mg) was chosen based on prior trial findings showing high efficacy of the 75 mg daily dose in suppressing viral shedding.

In a current phase III clinical trial, a 400 mg oral loading dose, followed by 100 mg daily oral dosing of pritelivir for up to 28 days or until all mucocutaneous HSV lesions are healed, whichever is earlier, is being investigated (https://www.clinicaltrials.gov/ct2/show/NCT03073967?term=pritelivir+f&draw=2&rank=2)

As is apparent from the above, the primary route of administration investigated for all of the small molecule based antiviral therapeutics described above is the oral route of administration. Efforts to improve therapy, e.g. to try and further reduce or prevent HSV shedding and viral reactivation, have largely focused on using higher oral drug doses (including high loading doses) and more frequent dosing (e.g. multiple daily dosing).

The focus on increasing systemic exposure via the oral route of administration is also evident from various attempts that have been made to improve the oral bioavailability of nucleoside analogues and helicase-primase inhibitors. For example, valacyclovir was developed as a pro-drug of acyclovir in order to provide higher oral bioavailability; the human bioavailability of valacyclovir following oral administration is approximately 54%, compared to only 10-20% for oral acyclovir (Murray (1995) Antiviral Chem. Chemotherapy 6(1), p. 34-38). For pritelivir, there has been a focus on salt forms with improved solubility for oral delivery. For example, WO 2013/045491 discloses that improved oral bioavailability can be achieved by using a crystalline mono-mesylate monohydrate salt of pritelivir with specific particle properties. The publication describes the free base of pritelivir as having unfavourable drug release and resorption properties due to its poor solubility when compared to the mono-mesylate monohydrate salt. The free base has also been characterised as being unsuitable for a long-term stable formulation. WO 2013/045479 discloses a particular crystalline mono-mesylate monohydrate salt of pritelivir and reports that the salt form has superior long term stability and improved systemic exposure following oral administration in comparison to pritelivir free base.

There is an ongoing need for novel and improved methods for treating HSV infections and the present invention was devised with the foregoing in mind.

### SUMMARY OF THE INVENTION

In a first aspect, the present invention provides a long-acting injectable depot pharmaceutical composition comprising a helicase-primase inhibitor or a pharmaceutically acceptable salt thereof; and one or more pharmaceutically acceptable excipients; wherein the helicase inhibitor has a human biological terminal half-life of 10 hours or more.

It has been surprisingly found that when a helicase-primase inhibitor with a human biological terminal half-life of 10 hours or more, or a pharmaceutically acceptable salt thereof, is formulated as a long-acting injectable depot pharmaceutical composition in accordance with the present invention, such a composition provides beneficial properties, including certain release characteristics and profiles that allow plasma levels to be achieved for prolonged periods.

Remarkably, in some embodiments, the long-acting injectable depot compositions of the present invention are able to provide steady and continuous release of a helicase-primase inhibitor for at least 14 days after administration. This offers the possibility for less frequent dosing and/or use of lower doses than other routes of administration. This is an unexpected finding. As described above, the primary route of administration investigated for small molecule based antiviral therapeutics is the oral route and efforts to improve therapy have largely focused on increasing oral drug doses (including the use of high loading doses) and using more frequent drug dosing regimens (e.g. multiple daily dosing).

In some embodiments, certain long-acting injectable depot compositions of the present invention have also been found to exhibit a high drug loading capacity. This provides a number of advantages, including the possibility of using low injection volumes to administer the compositions.

The present invention satisfies a need for a novel treatment approach for HSV infections which can provide improvements in efficacy and/or safety and/or patient use.

In a second aspect, the present invention provides a method for forming a pharmaceutical composition according to the first aspect. Various techniques may be used for forming the injectable depot compositions of the present invention, however, it has been found that certain processes offer particular advantages. Furthermore, it has also been found that certain chemical and/or physical forms of a helicase-primase inhibitor (e.g., forms with low aqueous solubility and low dissolution rates) can be highly suitable for formulation as injectable depot compositions of the present invention.

In a third aspect, the present invention provides a method for treating or preventing a herpes infection in a subject in need thereof, the method comprising administering a therapeutically effective amount of a helicase-primase inhibitor or a pharmaceutically acceptable salt thereof, to the subject, wherein the helicase-primase inhibitor or a pharmaceutically acceptable salt thereof, is formulated as a pharmaceutical composition according to the first aspect.

### BRIEF DESCRIPTION OF THE FIGURES

The summary, as well as the following detailed description, is further understood when read in conjunction with the appended figures. For the purpose of illustrating the disclosed compositions and methods, there are shown in the figures exemplary embodiments of the compositions and methods; however, the compositions and methods are not limited to the specific embodiments disclosed. In the figures:

FIG. 1 shows the XRPD diffractogram of the dihydrate of pritelivir free base.

FIG. 2 shows the mean plasma concentration-time profiles of pritelivir after sub-cutaneous dosing of the aqueous suspension formulation disclosed in Example 1 at 33.3 (open triangles) and 166.7 (open squares) mg/kg in male Sprague Dawley rats (n=3/arm).

FIG. 3 shows the mean plasma concentration-time profile of pritelivir after sub-cutaneous dosing of the aqueous suspension formulation disclosed in Example 2 at 10 mg/kg in male Sprague Dawley rats (n=3).

FIG. 4 shows the mean plasma concentration-time profiles of pritelivir after sub-cutaneous dosing of the aqueous suspension formulation disclosed in Example 3 at 10 mg/kg in male cynomolgus monkeys (n=3).

FIG. 5 shows the mean plasma concentration-time profiles of pritelivir after sub-cutaneous dosing of the formulation disclosed in Example 4 at 10 mg/kg in male non-naïve Beagle dogs (n=3).

FIG. 6 shows the mean plasma concentration-time profiles of pritelivir after intravenous (IV) dosing at 1 mg/kg and sub-cutaneous (SC) dosing at 5 mg/kg of the solution formulation disclosed in Example 5 in male Sprague Dawley rats (n=3/arm).

FIG. 7 shows the mean plasma concentration-time profile of Example 7 Compound 1 after sub-cutaneous dosing of the aqueous suspension formulation disclosed in Example 7 (triangles) at 10 mg/kg in male Sprague Dawley rats (n=3).

FIG. 8 shows the mean plasma concentration-time profiles of Example 7 Compound 1 after sub-cutaneous dosing of the aqueous suspension formulation disclosed in Example 7 (triangles) at 10 mg/kg in male cynomolgus monkeys (n=3).

FIG. 9 shows the mean plasma concentration-time profiles of Example 7 Compound 1 after sub-cutaneous dosing of the aqueous suspension formulation disclosed in Example 7 (triangles) at 10 mg/kg in male non-naïve Beagle dogs (n=3).

FIG. 10 shows the mean plasma concentration-time profile of Compound 1 (Form A) after sub-cutaneous dosing of the aqueous suspension formulation disclosed in Example 10 (triangles) at 10 mg/kg in male Sprague Dawley rats (n=3).

FIG. 11 shows the mean plasma concentration-time profiles of Compound 1 (Form A) after sub-cutaneous dosing of the aqueous suspension formulation disclosed in Example 11 (triangles) at 10 mg/kg in male cynomolgus monkeys (n=3).

FIG. 12 shows the mean plasma concentration-time profiles of Compound 1 (Form A) after sub-cutaneous dosing of the aqueous suspension formulation disclosed in Example 12 (diamonds) at 10 mg/kg in male non-naïve Beagle dogs (n=3).

FIG. 13 shows the mean plasma concentration-time profiles of Compound 1 after intra-venous dosing of the solution formulations disclosed in Example 13 (open triangles) at 1 mg/kg in male Sprague Dawley rats (n=3).

FIG. 14 shows the mean plasma concentration-time profiles of Compound 1 after intra-venous dosing of the solution formulations disclosed in Example 13 (open triangles) at 0.25 mg/kg in male cynomolgus monkeys (n=3).

FIG. 15 shows the mean plasma concentration-time profiles of Compound 1 after intra-venous dosing of the solution formulations disclosed in Example 13 (open triangles) at 0.15 mg/kg in male non-naïve Beagle dogs (n=3).

FIG. 16 shows the XRPD diffractogram of the sesquihydrate pritelivir free base.

FIG. 17 shows the XRPD diffractogram of anhydrous Form A of Compound 1 free form.

FIG. 18 shows the XRPD diffractogram of anhydrous Form C of Compound 1 free form.

FIG. 19 shows the mean plasma concentration-time profiles of Compound 1 after IV dosing of the solution formulations disclosed in Example 13A to (A) male Sprague Dawley rats (n=3) at 0.2 mg/kg; (B) male non-naïve Beagle dogs (n=3) at 0.15 mg/kg; (C) male non-naïve cynomolgus monkeys (n=3) at 0.2 mg/kg; and (D) naïve Bama mini-pigs (n=3) at 0.25 mg/kg.

FIG. 20 shows a SEM image of the micro-suspension described in Example 14.

FIG. 21 shows a SEM image of the nano-suspension described in Example 14.

FIG. 22 shows the mean plasma concentration-time profiles of pritelivir after sub-cutaneous dosing of the solution (closed circles), microsuspension (closed triangles) and nanosuspension (closed squares) formulations disclosed in Example 14 at 10 mg/kg in male non-naïve Beagle dogs (n=3).

FIG. 23 shows the mean plasma concentration-time profile of Compound 1 after sub-cutaneous dosing of the solution formulation disclosed in Example 15 (closed triangles) at 100 mg/kg in male Sprague Dawley rats (n=3).

FIG. 24 shows the mean plasma concentration-time profiles of Compound 1 after sub-cutaneous dosing of the solution formulations disclosed in Example 16 at 10 mg/kg; 0.1 ml/kg (closed triangles) and 10 mg/kg; 0.2 ml/kg (closed diamonds) in male non-naïve Beagle dogs (n=3).

FIG. 25 shows a SEM image of the micro-suspension described in Example 17.

FIG. 26 shows a SEM image of the nano-suspension described in Example 17.

FIG. 27 shows the mean plasma concentration-time profile of Compound 1 (Form C) after sub-cutaneous dosing of the microsuspension (closed squares) and nanosuspension (closed triangles) formulations disclosed in Example 17 at 20 mg/rat (−80 mg/kg) in male Sprague Dawley rats (n=3).

FIG. 28 shows the mean plasma concentration-time profile of Compound 1 (Form C) after intra-muscular dosing of the microsuspension (closed squares) and nanosuspension (closed triangles) formulations disclosed in Example 17 at 20 mg/rat (−80 mg/kg) in male Sprague Dawley rats (n=3).

### DETAILED DESCRIPTION OF THE INVENTION

The disclosed compositions, uses thereof and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

### Definitions

Unless otherwise stated, the following terms used in the specification and claims have the following meanings set out below.

Reference to a particular numerical value includes at least that particular value unless the context clearly dictates otherwise. When a range of values is expressed, another embodiment includes from the one particular value and/or to the other particular value. Further, reference to values stated in ranges include each and every value within that range. All ranges are inclusive and combinable.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any sub-combination.

As used herein, the singular forms “a,” “an,” and “the” include the plural.

As used herein, Cₘₐₓ refers to the geometric mean maximum concentration of the active agent. This may be measured in vivo following administration of a composition of the invention to a subject and measuring the plasma levels of the drug at various timepoints after dosing.

As used herein, AUC refers to the area under the curve and is the definite integral of the concentration of the active agent in blood plasma as a function of time.

As used herein, “patient” or “subject” refers to a mammal, including a domestic animal, animal kept as livestock and a zoo animal. Conveniently, the “patient” or “subject” is a human being.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms. As used herein, “treating” and like terms may specifically include reducing the severity and/or frequency of HSV induced symptoms, eliminating HSV induced symptoms and/or the underlying cause of said symptoms, reducing the frequency or likelihood of HSV induced symptoms and/or their underlying cause, delaying, preventing and/or slowing the progression of HSV induced conditions, and improving or remediating damage caused, directly or indirectly, by HSV infections. The term “preventing,” as used herein with respect to an HSV infection or HSV-related disorder, refers to reducing the likelihood of HSV infection. In one embodiment, “treating” or “treatment” of a state, disorder or condition means inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms. Conveniently, “treating” and like terms mean reducing the severity and/or frequency of HSV induced symptoms, eliminating HSV induced symptoms and/or the underlying cause of said symptoms, reducing the frequency or likelihood of HSV induced symptoms and/or their underlying cause, delaying, preventing and/or slowing the progression of HSV induced conditions, and/or improving or remediating damage caused, directly or indirectly, by HSV infections.

A “therapeutically effective amount” or “therapeutically effective dose” means the amount of a compound that, when administered to a patient or subject for treating a disease, is sufficient to effect such treatment for the disease. As used herein, the phrase “therapeutically effective dose” or “therapeutically effective amount” may specifically refer to the amount of a helicase-primase inhibitor dosed to a patient or subject using a long-acting injectable pharmaceutical composition, as described herein, which is effective to achieve a particular biological or therapeutic result such as, but not limited to, biological or therapeutic results disclosed, described, or exemplified herein. The therapeutically effective dose may vary according to factors such as the disease state, age, sex, and weight of the individual, and the ability of the composition to cause a desired response in a subject. Such results include, but are not limited to, the reduction, remission, and/or regression of conditions caused by, or associated with, HSV or prevention of the development of conditions caused by, or associated with, HSV, as determined by any means suitable in the art.

When values are expressed as approximations, by use of the antecedent “about,” it will be understood that the particular value forms another embodiment. Further, the term “about” refers to a ±10% variation from the nominal value unless otherwise indicated or inferred.

The term “about” when used in reference to numerical ranges, cut-offs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

At various places in the present specification, values are disclosed in groups or in ranges. It is specifically intended that the description include all individual sub-combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

As used herein, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

The use of any and all examples, or exemplary language herein, for example, “such as,” “including,” or “for example,” is intended merely to illustrate better the present teachings and does not pose a limitation on the scope of the invention unless claimed. No language in the specification should be construed as indicating any non-claimed element as essential to the practice of the present teachings.

### Helicase-Primase Inhibitor

The expression “helicase-primase inhibitor” within the context of the present invention denotes any compound or agent able to reduce viral replication by inhibiting the viral complex consisting of DNA helicase, DNA primase, and cofactor subunits. The helicase-primase complex is used by herpes viruses, therefore the helicase-primase inhibitor will have antiviral activity against one or more herpes viruses, such as one or more of Herpes Simplex Virus subtype type 1 and 2 (HSV-1, HSV-2), Varicella Zoster Virus (VZV), Epstein-Barr virus (EBV), Cytomegalovirus (CMV), and Human Herpes Viruses 6-8 (HHV 6-8).

In an embodiment, the helicase-primase inhibitor has antiviral activity against HSV-1 and/or HSV-2.

In an embodiment, the helicase-primase inhibitor has an in-vitro EC₅₀ value of less than about 0.1 μM against HSV-1 and/or HSV-2. Conveniently, the helicase-primase inhibitor has an in-vitro EC₅₀ value of less than about 0.075 μM or less than about 0.05 μM against HSV-1 and/or HSV-2, conveniently against HSV-1 and HSV-2. More conveniently, the helicase-primase inhibitor has an in-vitro EC₅₀ value of less than about 0.030 μM against HSV-1 and/or HSV-2. More conveniently, the helicase-primase inhibitor has an in-vitro EC₅₀ value of less than about 0.020 μM against HSV-1 and/or HSV-2. The in-vitro EC₅₀ value against HSV-1 and/or HSV-2 can be determined in accordance with methods known to the skilled person, such as those disclosed in Field et al. (2013, Antiviral Res. 100, p. 297-299). The in-vitro EC₅₀ value can also be determined in accordance with the assays described in the Examples section of the present application.

In vivo the helicase-primase inhibitor may be subject to plasma protein binding. In an embodiment, the EC₅₀ value of the helicase-primase inhibitor (such as pritelivir) may correspond to the unbound plasma concentration of the active agent.

In an embodiment, the helicase-primase inhibitor shows no or low levels of carbonic anhydrase inhibition, such as inhibition of carbonic anhydrase I and/or carbonic anhydrase II. Carbonic anhydrase inhibition can be measured using a carbonic anhydrase I assay as described in Katritzky et al. (J. Med. Chem. 1987, 30:2058) and a carbonic anhydrase I assay as described in lyer et al. (J. Biomol. Screen 2006, 11:782). In an embodiment, the helicase-primase inhibitor has an IC₅₀ of greater than 2.0 μM, greater than 3.0 μM, and most conveniently, greater than 5.0 μM when measured in either or both of these assays.

The helicase-primase inhibitor used in the compositions according to the present invention has a human biological terminal half-life of 10 hours or more. The human biological terminal half-life of a helicase-primase inhibitor could be derived from the terminal elimination phase of the pharmacokinetic profile following intravenous administration of the helicase-primase inhibitor. Pritelivir has a human biological terminal half-life of up to 80 hours (Wald et al., (2014) New England Journal of Medicine 370, p. 201-210). In an embodiment, the helicase-primase inhibitor has a human biological terminal half-life of 20 hours or more, such as 30 hours or more, 40 hours or more, 50 hours or more, 60 hours or more, 70 hours or more, or 80 hours or more. Conveniently, the helicase-primase inhibitor has a human biological terminal half-life of 30 hours or more.

In an embodiment, the helicase-primase inhibitor or pharmaceutically acceptable salt thereof has a low aqueous solubility. Conveniently, the compound has a solubility in water or an aqueous solvent system (measured at room temperature) of less than 100 μg/ml, of less than 50 μg/ml, suitably less than 25 μg/ml, more suitably, less than 20 μg/ml. Conveniently, the compound has a solubility in water (measured at room temperature) at about pH 7.0 of less than 100 μg/ml, of less than 50 μg/ml, suitably less than 25 μg/ml, more suitably, less than 20 μg/ml.

Examples of helicase-primase inhibitors include:
N-Methyl-N-(4-methyl-5-sulfamoyl-1,3-thiazol-2-yl)-2-[4-(pyridin-2-yl)phenyl]acetamide (pritelivir; WO 01/47904);
(S)-2-(2′,5′-difluoro-[1,1′-biphenyl]-4-yl)-N-methyl-N-(4-methyl-5-(S-methylsulfon-imidoyl)thiazol-2-yl)acetamide (IM-250; WO 2017/174640 and WO2019/068817A1); and
N-[2-[4-(2-aminothiazol-4-yl)anilino]-2-oxo-ethyl]-N-[(1S)-1-phenylethyl]pyridine-4-carboxamide (BILS-179BS; WO 97/24343).

The helicase-primase inhibitor of the present invention comprises both a parent compound and any pharmaceutically acceptable salt of the parent compound.

In cases where a helicase-primase inhibitor is sufficiently basic or acidic to form stable pharmaceutically acceptable acid or base salts, preparation and administration of the helicase-primase inhibitor as a pharmaceutically acceptable salt may be appropriate.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Examples of pharmaceutically acceptable salts are inorganic and organic acid addition salts formed with acids which form a physiological acceptable anion, for example, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Helicase-primase inhibitors that are acidic in nature may be capable of forming base salts with various pharmacologically acceptable cations. Examples of such salts include alkali metal or alkaline earth metal salts, particularly calcium, magnesium, sodium, lithium, zinc, potassium, and iron salts.

It is also to be understood that certain inhibitors of the invention may exhibit polymorphism, and that the invention encompasses all such forms (including anhydrous/non-solvated forms, solvates and hydrates).

In an embodiment the helicase-primase inhibitor is present in the composition as a crystalline solid. A crystalline solid form is a solid material wherein the constituents of the solid material are arranged in a highly ordered microscopic structure, thereby forming a crystal lattice which extends in all directions. The crystalline solid may be present in any suitable form, such as anhydrate, hydrate or solvate forms. It is also to be understood that the helicase-primase inhibitor of the present invention may be pure, essentially pure, or have a purity level greater than 75%, such as greater than 85%, greater than 90%, greater than 95%, greater than 97%, greater than 98%, or greater than 99% with respect to other crystalline forms (for examples as measured by X-Ray powder diffraction).

It has been found that certain crystalline solid forms of a helicase-primase inhibitor, e.g. certain crystalline solid forms of pritelivir, are surprisingly well suited to the long-acting injectable depot compositions of the present invention.

In an embodiment the helicase-primase inhibitor is pritelivir, or a pharmaceutically acceptable salt thereof. Pritelivir is N-methyl-N-(4-methyl-5-sulfamoyl-1,3-thiazol-2-yl)-2-[4-(pyridin-2-yl)phenyl]acetamide having the following structure:

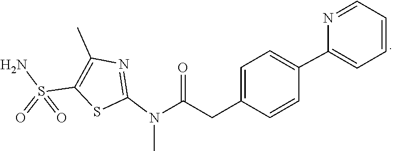 [SMILES: Cc1nc(N(C)C(=O)Cc2ccc(-c3ccccn3)cc2)sc1S(N)(=O)=O]

It is understood that pritelivir has potent antiviral activity against HSV-1 and HSV-2 with in-vitro EC₅₀ values of 0.026 μM and 0.029 μM respectively (Field et al., (2013) Antiviral Res. 100, p. 297-299).

In an embodiment, the helicase-primase inhibitor is pritelivir free base. The free base of pritelivir has a low aqueous solubility and has been dismissed as being unsuitable for use in providing a long-term stable formulation (WO 2013/045491). However, the current applicants have found that the free base compound is particularly suited for formulation in a long-acting injectable depot composition in accordance with the present invention.

In a convenient embodiment the helicase-primase inhibitor is pritelivir free base and the free base is present in the composition as a crystalline solid. In an embodiment, the pritelivir crystalline free base is an anhydrous (anhydrate) or hydrated form.

Conveniently, the pritelivir crystalline free base is a hydrate. Conveniently, the pritelivir crystalline free base is a hemihydrate, dihydrate or a sesquihydrate. More conveniently, the pritelivir crystalline free base is a dihydrate or a sesquihydrate. Most conveniently, the pritelivir crystalline free base is a sesquihydrate as disclosed in the Examples.

In an embodiment, the helicase-primase inhibitor is Compound 1 represented by:

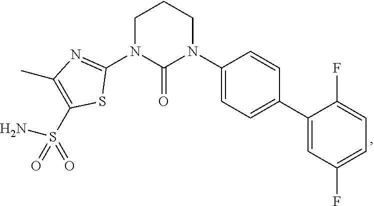 [SMILES: Cc1nc(N2CCCN(c3ccc(-c4cc(F)ccc4F)cc3)C2=O)sc1S(N)(=O)=O]

or a pharmaceutically acceptable salt thereof.

The compound shown above is 2-(3-(2′,5′-difluoro-[1,1′-biphenyl]-4-yl)-2-oxotetrahydropyrimidin-1(2H)-yl)-4-methylthiazole-5-sulfonamide (also referred to herein as ‘Compound 1’) and is a potent helicase-primase inhibitor.

Compound 1 has an in-vitro EC₅₀ value of approximately 0.019 μM against HSV-1 and 0.011 μM against HSV-2. The in-vitro EC₅₀ value against HSV-1 and/or HSV-2 can be determined in accordance with methods known to the skilled person, such as those disclosed in Field et al. (2013, Antiviral Res. 100, p. 297-299). The in-vitro EC₅₀ value can also be determined in accordance with the assays described in the Examples section of the present application.

Compound 1 has been found to have a predicted human biological terminal half-life of over 180 hours (see Examples 13 and 13A).

Compound 1 shows no or low levels of carbonic anhydrase inhibition, such as inhibition of carbonic anhydrase I and/or carbonic anhydrase II. Carbonic anhydrase inhibition can be measured using a carbonic anhydrase I assay as described in Katritzky et al. (J. Med. Chem. 1987, 30:2058) and a carbonic anhydrase I assay as described in lyer et al. (J. Biomol. Screen 2006, 11:782).

It is to be understood that Compound 1 may exhibit polymorphism, and that the invention encompasses all such forms (including anhydrous/non-solvated forms, solvates and hydrates).

In an embodiment Compound 1 is present in the composition as a crystalline solid. The crystalline solid may be present in any suitable form, such as anhydrate, hydrate or solvate forms. It is also to be understood that Compound 1 may be pure, essentially pure, or have a purity level greater than 75%, such as greater than 85%, greater than 90%, greater than 95%, greater than 97%, greater than 98%, or greater than 99% with respect to other crystalline forms (for example as measured by X-Ray powder diffraction).

In a convenient embodiment the helicase-primase inhibitor present in the compositions of the invention is Compound 1 free form and the free form is present in the composition as a crystalline solid. In a convenient embodiment, the Compound 1 crystalline free form is an anhydrous form. It has been found that certain crystalline solid forms of the helicase-primase inhibitor, Compound 1, are surprisingly well suited to the long-acting injectable depot compositions of the present invention. Conveniently, the Compound 1 crystalline free form is Form A or Form C, as described in the Examples. More conveniently, the Compound 1 crystalline free form is Form C, as described in the Examples.

### Pharmaceutical Compositions of the Invention

In a first aspect, the present invention provides a long-acting injectable depot pharmaceutical composition comprising a helicase-primase inhibitor or a pharmaceutically acceptable salt thereof; and one or more pharmaceutically acceptable excipients; wherein the helicase-primase inhibitor has a human biological terminal half-life of 10 hours or more.

As used herein, “long-acting injectable depot pharmaceutical composition”, refers to a pharmaceutical composition, which a formulation adapted for administration to a subject (conveniently a human) via the subcutaneous or intramuscular route, wherein upon administration, a depot is formed from which the active ingredient of the composition (helicase-primase inhibitor) is released over a prolonged period of time.

In an embodiment, prolonged release refers to continuous active ingredient release over a period of at least 10 days, such as at least 14 days, at least 21 days, at least 28 days, at least 56 days, at least 72 days, at least 96 days, at least 120 days, at least 180 days, at least 240 days, or at least 300 days. The prolonged release of the active ingredient means that the active ingredient plasma levels may be kept at a therapeutically effective plasma concentration for at least 10 days, such as at least 14 days, at least 21 days, at least 28 days, at least 56 days, at least 72 days, at least 96 days, at least 120 days, at least 180 days, at least 240 days, or at least 300 days after administration of the composition, which a person skilled in the art will understand corresponds to “long-acting”.

It is to be understood that the rate of release of the helicase-primase may vary during the prolonged release period, for example a short “initial burst” of active agent may be observed shortly after administration followed by a period of lower release. However, prolonged release means that plasma levels of the of the helicase-primase inhibitor can be kept at therapeutically effective plasma concentrations throughout most if not all of the duration of the prolonged release period.

In one embodiment, the pharmaceutical composition provides a short “initial burst” of active agent shortly after administration followed by a period of lower release. Advantageously, the applicants have found that certain compositions of the invention are able to provide a rapid initial release to achieve a high plasma levels of the helicase-primase inhibitor, followed by continuous release to maintain plasma levels at therapeutically effective concentrations over a prolonged period of time.

In an embodiment, the pharmaceutical composition releases less than 20% (conveniently less than 10%), by weight of the total amount of the active agent contained within the pharmaceutical composition within a period of 24 hours (conveniently 48 hours) after administration.

Advantageously, the applicants have found that the prolonged release provided by certain compositions of the invention, and the resultant continuous plasma levels at therapeutically effective concentration over a prolonged period allow the possibility of using lower than expected doses of the helicase-primase inhibitor. In an embodiment, the dose administered in a single administration of a long-acting injectable depot pharmaceutical composition, which is capable of providing prolonged release over a period of at least 14 days (conveniently at least 28 days, at least 56 days, at least 96 days, at least 120 days, at least 180 days, at least 240 days, or at least 300 days), is about 100 to 1200 mg (conveniently 300 to 900 mg) of the helicase-primase inhibitor or a pharmaceutically acceptable salt thereof.

In an embodiment, the dose administered in a single administration of a long-acting injectable depot pharmaceutical composition, which is capable of providing prolonged release over a period of at least 1 month, is about 100 to 900 mg (conveniently 100 to 600 mg) of the helicase-primase inhibitor or a pharmaceutically acceptable salt thereof.

In an embodiment, the dose administered in a single administration of a long-acting injectable depot pharmaceutical composition, which is capable of providing prolonged release over a period of at least 2 months, is about 200 to 900 mg (conveniently 200 to 600 mg) of the helicase-primase inhibitor or a pharmaceutically acceptable salt thereof.

In an embodiment, the dose administered in a single administration of a long-acting injectable depot pharmaceutical composition, which is capable of providing prolonged release over a period of at least 3 months, is about 300 to 900 mg (conveniently 300 to 600 mg) of the helicase-primase inhibitor or a pharmaceutically acceptable salt thereof.

In an embodiment, the dose administered in a single administration of a long-acting injectable depot pharmaceutical composition, which is capable of providing prolonged release over a period of at least 6 months, is about 300 to 1200 mg (conveniently 300 to 900 mg) of the helicase-primase inhibitor or a pharmaceutically acceptable salt thereof.

In some embodiments, certain long-acting injectable depot compositions of the present invention have been found to exhibit a high drug loading capacity. This provides a number of advantages, including the possibility of administering the compositions in low injection volumes. In an embodiment, the dose is administered in a single administration of a long-acting injectable depot pharmaceutical composition in an injection volume of about 0.1 to about 5 mL. Conveniently the dose is administered in an injection volume of about 0.1 to about 3 mL, about 0.1 to about 1.5 mL, or about 0.1 to about 0.75 mL. More conveniently, the dose is administered in an injection volume of about 0.1 to about 0.5 mL.

In a convenient embodiment, the pharmaceutical composition is designed for subcutaneous or intramuscular injection. In a further aspect of the present invention, the pharmaceutical composition is for subcutaneous administration. In a yet further aspect of the present invention, the pharmaceutical composition is for intramuscular administration. Conveniently, the dose of the helicase-primase inhibitor is administered subcutaneously in an injection volume of about 0.1 to about 3 mL, or about 0.1 to about 1.5 mL. Conveniently, the dose of the helicase-primase inhibitor is administered intramuscularly in an injection volume of about 0.1 to about 5 mL, or about 0.1 to about 3 mL.

The benefits of the present invention are not limited to a particular type of long-acting injectable depot pharmaceutical composition having a particular mechanism of drug release. The pharmaceutical composition adapted for subcutaneous and/or intramuscular administration can be an aqueous based depot, an aqueous based suspension depot, a solution in organic solvent based formulation, a suspension in organic solvent based depot, a gel based depot, an in-situ gelling depot formulation, an oil-based depot, an emulsion based depot, a monolithic polymer based depot, a microparticle polymer based depot or a solid implant depot.

In an embodiment, the pharmaceutical composition of the present invention comprises a rate-controlling agent to help provide prolonged release of the helicase-primase inhibitor. In an embodiment, the rate-controlling agent comprises at least one biodegradable polymer. Suitable biodegradable polymers are polymers capable of breaking down under physiological conditions such that the polymer and its degradation products do not invoke any unacceptable toxicity or immune response. Such polymers may be natural or synthetic in origin. Examples of natural biodegradable polymers include polysaccharides such as chitosan, alginate and dextran, or polyesters such as polyhydroxyalkanoates. Examples of synthetic biodegradable polymers include polymers of lactic acid and glycolic acid and copolymers thereof. In an embodiment, the biodegradable polymer is poly(lactic-co-glycolic acid) or polylactic acid, wherein each polymer is end-capped with either acid or ester groups. Conveniently, the biodegradable polymer is poly(lactic-co-glycolic acid). Poly(lactic-co-glycolic acid) is referred to herein also as PLGA. Conveniently, the biodegradable polymer is PLGA end-capped with acid groups. In an embodiment, the biodegradable polymer is PLGA, wherein the PLGA has a lactic acid weight content of about 5 to 95% with the balance being glycolic acid.

In a convenient embodiment, the pharmaceutical composition of the present invention is an aqueous based suspension, a solution in organic solvent-based formulation, or an in-situ gelling depot formulation.

In a preferred embodiment, the pharmaceutical composition of the present invention is an aqueous based suspension depot. Surprisingly, it has been found that certain aqueous based suspension depot compositions provide active ingredient plasma levels at therapeutically effective plasma concentrations for prolonged periods without the need for a rate-controlling agent. This can be particularly advantageous as it allows high drug loading, smaller administration volumes and lower excipient levels. In an embodiment, the pharmaceutical composition of the present invention does not comprise a rate-controlling agent or contains less than 20% or less than 15, 10, 5, 3, 2 or 1% by weight of the total composition of a rate-controlling agent. Conveniently, the pharmaceutical composition of the present invention does not comprise a rate-controlling agent, such as a biodegradable polymer.

It has also been found that release can be influenced by the particle size of the helicase-primase inhibitor in an aqueous based suspension depot composition. Suitable methods to determine the particle size distribution of a sample will be apparent to a person of skill in the art and include techniques such as laser diffraction. The term Dx (or Dvₓ) refers to the particle size up to and including which x % of the total volume of material in the sample is contained. For example, a D50 (or Dv₅₀) of 10 μm, means that 50% of the sample has a particle size of 10 μm or smaller.

In an embodiment, the particle size volume distribution of the suspended particles of active agent (helicase-primase inhibitor) in an aqueous based suspension depot composition are such that less than 10% of the particles have a size greater than 9 microns, when measured by laser diffraction analysis. In an embodiment, the aqueous based suspension depot composition comprises particles of active agent having a D10 of 0.5 to 2.5 μm, a D50 of 2 to 5 μm, and/or a D90 of 4 to 13 μm. In an embodiment, the aqueous based suspension depot composition comprises particles of active agent having a D10 of 0.5 to 2.5 μm, a D50 of 3 to 5 μm, and/or a D90 of 8 to 13 μm. In an embodiment, the aqueous based suspension depot composition comprises particles of active agent having a D10 of 0.5 to 2.0 μm, a D50 of 2 to 3 μm, and/or a D90 of 4 to 8 μm. In an embodiment, the aqueous based suspension depot composition comprises particles of active agent having a D10 of 1 to 2 μm, a D50 of 4 to 5 μm, and/or a D90 of 10 to 11 μm.

In an embodiment, the aqueous based suspension depot composition comprises particles of active agent having a D90 of 10 to 5000 nm, such as 20 to 2000 nm, or 50 to 1000 nm. Conveniently, the helicase-inhibitor is pritelivir.

In an embodiment, the helicase-primase inhibitor is Compound 1 and the aqueous based suspension depot composition comprises particles of active agent having a D10 of 0.3 to 1.5 μm, a D50 of 2 to 4 μm, and/or a D90 of 8 to 16 μm. In an embodiment, the aqueous based suspension depot composition comprises particles of active agent having a D10 of 0.3 to 0.7 μm, a D50 of 2 to 3 μm, and/or a D90 of 12 to 16 μm.

In an embodiment, the helicase-primase inhibitor is pritelivir and the pharmaceutical composition is an aqueous based microsuspension depot composition. Conveniently, the particle size volume distribution of the suspended particles of pritelivir in the aqueous based microsuspension depot composition are such that less than 10% of the particles have a size greater than 20 μm, when measured by laser diffraction analysis. In an embodiment, the aqueous based microsuspension depot composition comprises particles of pritelivir having a D90 of 4 to 20 μm, conveniently 5 to 15 μm, more conveniently 6 to 10 μm. In an embodiment, the aqueous based microsuspension depot composition comprises particles of pritelivir having a D10 of 0.3 to 3 μm, a D50 of 2 to 6 μm, and/or a D90 of 5 to 15 μm. In an embodiment, the aqueous based microsuspension depot composition comprises particles of pritelivir having a D10 of 1.5 to 2.5 μm, a D50 of 3 to 5 μm, and/or a D90 of 6 to 10 μm.

In an embodiment, the helicase-primase inhibitor is pritelivir and the pharmaceutical composition is an aqueous based nanosuspension depot composition. Conveniently, the particle size volume distribution of the suspended particles of pritelivir in the aqueous based nanosuspension depot composition are such that less than 10% of the particles have a size greater than 700 nm, when measured by laser diffraction analysis. In an embodiment, the aqueous based nanosuspension depot composition comprises particles of pritelivir having a D90 of 1 to 700 nm, conveniently 1 to 500 nm, more conveniently 1 to 300 nm.

In an embodiment, the helicase-primase inhibitor is Compound 1 and the pharmaceutical composition is an aqueous based microsuspension depot composition. Conveniently, the particle size volume distribution of the suspended particles of Compound 1 in the aqueous based microsuspension depot composition are such that less than 10% of the particles have a size greater than 15 μm, when measured by laser diffraction analysis. In an embodiment, the aqueous based microsuspension depot composition comprises particles of Compound 1 having a D90 of 4 to 13 μm, conveniently 4 to 10 μm, more conveniently 4 to 8 μm. In an embodiment, the aqueous based microsuspension depot composition comprises particles of Compound 1 having a D10 of 0.3 to 2 μm, a D50 of 2 to 4 μm, and/or a D90 of 4 to 13 μm. In an embodiment, the aqueous based microsuspension depot composition comprises particles of Compound 1 having a D10 of 0.3 to 1.5 μm, a D50 of 2 to 3.5 μm, and/or a D90 of 4 to 8 μm.

In an embodiment, the helicase-primase inhibitor is Compound 1 and the pharmaceutical composition is an aqueous based nanosuspension depot composition.

Conveniently, the particle size volume distribution of the suspended particles of Compound 1 in the aqueous based nanosuspension depot composition are such that less than 10% of the particles have a size greater than 500 nm, when measured by laser diffraction analysis. In an embodiment, the aqueous based nanosuspension depot composition comprises particles of Compound 1 having a D90 of 1 to 400 nm, conveniently 1 to 300 nm, more conveniently 1 to 200 nm. In an embodiment, the aqueous based nanosuspension depot composition comprises particles of Compound 1 having a D10 of 1 to 100 nm, a D50 of 1 to 200 nm, and/or a D90 of 1 to 300 nm. In an embodiment, the aqueous based nanosuspension depot composition comprises particles of Compound 1 having a D10 of 1 to 100 nm, a D50 of 1 to 150 nm, and/or a D90 of 1 to 200 nm. In an embodiment, the aqueous based nanosuspension depot composition comprises particles of Compound 1 having a D10, D50 and D90 of 1 to 100 nm.

In an embodiment, the pharmaceutical composition of the present invention is a solution in organic solvent based formulation. The organic solvent may be any suitable organic solvent that solubilises the helicase-primase inhibitor and is biocompatible. The suitable biocompatible organic solvent, after the formulation has been injected in-vivo, diffuses from the formulation leading to solidification of the formulation. Conveniently, the biocompatible solvent is non-toxic, water miscible, and does not cause severe tissue irritation or necrosis at the site of injection/implantation. Examples of such solvents may include benzyl alcohol, benzyl benzoate, N-methyl-2-pyrrolidone, 2-pyrrolidone, ethanol, propylene glycol, acetone, methyl acetate, ethyl acetate, methyl ethyl ketone, dimethylacetamide, dimethylformamide, dimethyl sulfoxide, tetrahydrofuran, caprolactam, oleic acid, and I-dodecylazacycloheptan-2-one. In an embodiment, the organic solvent is selected from N-methyl pyrrolidone (NMP), dimethylacetamide (DMA), dimethyl sulfoxide (DMSO), benzyl alcohol, benzyl benzoate and mixtures thereof. The solution in organic solvent based formulation may optionally further comprise an aqueous media (such as aqueous buffer or saline) and/or a surfactant (such as a polyethylene glycol fatty ester, e.g. PEG-15 hydroxystearate).

In an embodiment, the helicase-primase inhibitor is Compound 1 and the pharmaceutical composition is a solution in organic solvent based formulation. In an embodiment, the solution in organic solvent based formulation comprises Compound 1 dissolved in an organic solvent selected from N-methyl pyrrolidone (NMP), dimethylacetamide (DMA), dimethyl sulfoxide (DMSO), benzyl alcohol, benzyl benzoate and mixtures thereof, preferably N-methyl pyrrolidone (NMP), dimethylacetamide (DMA), and dimethyl sulfoxide (DMSO), such as NMP. In an embodiment, the solution in organic solvent based formulation comprising Compound 1 may optionally further comprise an aqueous media (such as aqueous buffer or saline) and/or a surfactant (such as a polyethylene glycol fatty ester, e.g. PEG-15 hydroxystearate). In an embodiment, the composition comprises 5-95% v/v (such as 5-75%, 5-50%, 5-20%, or 5-15% v/v) of an organic solvent selected from N-methyl pyrrolidone (NMP), dimethylacetamide (DMA), dimethyl sulfoxide (DMSO), and mixtures thereof. In an embodiment, the composition comprises Compound 1 at a concentration of 1-500 mg/mL, such as 10-400, 100-400, or 200-300 mg/mL. In an embodiment, the composition comprises Compound 1 at a concentration of 100-400 mg/mL and 5-95% v/v of NMP as the organic solvent.

In an embodiment, the helicase-primase inhibitor is pritelivir free base and the pharmaceutical composition is a solution in organic solvent based formulation. In an embodiment, the solution in organic solvent based formulation comprises Compound 1 dissolved in an organic solvent selected from N-methyl pyrrolidone (NMP), dimethylacetamide (DMA), dimethyl sulfoxide (DMSO), benzyl alcohol, benzyl benzoate and mixtures thereof, preferably N-methyl pyrrolidone (NMP), dimethylacetamide (DMA), and dimethyl sulfoxide (DMSO). In an embodiment, the solution in organic solvent based formulation comprising pritelivir free base may optionally further comprise an aqueous media (such as aqueous buffer or saline) and/or a surfactant (such as a polyethylene glycol fatty ester, e.g. PEG-15 hydroxystearate). In an embodiment, the composition comprises 5-95% v/v (such as 5-75%, 5-50%, 5-20%, or 5-15% v/v) of an organic solvent selected from N-methyl pyrrolidone (NMP), dimethylacetamide (DMA), dimethyl sulfoxide (DMSO), and mixtures thereof. In an embodiment, the composition comprises pritelivir free base at a concentration of 1-500 mg/mL, such as 10-400, 50-300, or 50-200 mg/mL. In an embodiment, the composition comprises pritelivir free base at a concentration of 10-400 mg/mL (such as 100-400 mg/mL) and 5-95% v/v of NMP as the organic solvent.

In an embodiment, the pharmaceutical composition of the present invention is an in-situ gelling depot formulation. The term “in situ gelling depot formulation” as used herein refers to a formulation comprising a helicase-primase inhibitor, a biodegradable polymer (such as the biodegradable polymers described as rate-controlling agents above) and a biocompatible solvent, which is delivered to a patient as an injectable liquid but solidifies into a solid depot formulation as the liquid solvent diffuses away in vivo. The suitable biocompatible solvent refers to any solvent in which the components of the formulation can be dissolved and which after the formulation has been injected in-vivo diffuses from the formulation leading to solidification of the formulation. Conveniently, the solvent for the biodegradable polymer be non-toxic, water miscible, and otherwise biocompatible. The solvents must also be biocompatible so that they do not cause severe tissue irritation or necrosis at the site of implantation. Furthermore, the solvent should be water miscible so that it will diffuse quickly into the body fluids and allow water to permeate into the polymer solution and cause it to coagulate or solidify. Examples of such solvents include benzyl alcohol, N-methyl-2-pyrrolidone, 2-pyrrolidone, ethanol, propylene glycol, acetone, methyl acetate, ethyl acetate, methyl ethyl ketone, dimethylformamide, dimethyl sulfoxide, tetrahydrofuran, caprolactam, oleic acid, and I-dodecylazacycloheptan-2-one.

One or more pharmaceutically acceptable excipients is included in the composition according to the present invention. The long-acting injectable depot pharmaceutical compositions of the invention comprise at least one excipient selected from the group consisting of a solvent, co-solvent, wetting or suspending agent, an isotonic agent, a pH adjusting agent, a stabiliser, an emulsifier and a viscosity modifier.

Suitable wetting or suspending agents, included in the formulations of the present invention, include cellulose derivatives, polyvinylpyrrolidone, polysorbate 20 and polysorbate 80, lecithin, polyoxyethylene sorbitan fatty acid esters and polyoxyethylene sorbitan fatty acid esters. Conveniently, the polysorbate 20 or are used alone or in combination. Conveniently, polysorbate 80 is present in the composition. Conveniently, the composition comprises about 0.2 wt % to about 5 wt % of polysorbate 80, more conveniently about 0.2 wt % to about 2 wt % of polysorbate 80.

Isotonic agents that can be included in the long-acting injectable depot pharmaceutical compositions of the present invention are, for example, sodium chloride, dextrose, mannitol, sorbitol, lactose, and/or sodium sulfate. Conveniently, the isotonic agent is sodium chloride. Conveniently, the isotonic agent is mannitol.

The pH adjusting agent contained in long-acting injectable depot pharmaceutical compositions of the present invention may be hydrochloric acid or sodium hydroxide, but is not limited thereto.

The depot preparation of the present invention may further contain a preservative and the preservative may be selected from the group consisting of benzoic acid, benzyl alcohol, butylated hydroxyanisole, butylated hydroxytoluene, chlorbutol, gallate, hydroxybenzoate, EDTA, phenol, parabens, methyl paraben, propyl paraben, butyl paraben, benzalkonium chloride, thiomerosal, meta-cresol or chlorobutanol, or the like.

In some embodiments, the long-acting injectable depot pharmaceutical compositions of the invention comprise at least one viscosity modifier. In some cases, the viscosity modifier is selected from the group consisting of sodium carboxymethyl cellulose, methyl cellulose, hydroxypropyl cellulose, calcium carboxymethyl cellulose, crosslinked carboxymethyl cellulose, hydroxyethylcellulose, hydroxypropylmethyl cellulose, polyvinyl alcohol, acacia, gelatin, and polyvinyl pyrrolidone. Conveniently, the Conveniently, the composition comprises about 0.1 wt % to about 5 wt % of the viscosity modifier. In some embodiments, the viscosity modifier is selected from the group consisting of sodium carboxymethyl cellulose and/or hydroxypropyl cellulose and/or polyvinyl pyrrolidone.

The long-acting injectable depot pharmaceutical compositions of the present invention may be stored by freeze-drying, e.g. in a suitable vial, and can be resuspended, for injection immediately before injection. Compositions can be stored in a pre-filled syringe.

Advantageously, the compositions of the invention may demonstrate good stability on storage. Therefore, in an embodiment, the composition is stable for at least four weeks, such as for at least eight weeks, or at least twelve weeks. In this context, ‘stable’ may refer to the physical and/or chemical stability of the active agent (helicase-primase inhibitor) within the long-acting injectable depot pharmaceutical composition. ‘Stable’ may also refer to the release profile or pharmacokinetics of the composition being maintained over the storage period referred to. ‘Stable’ may also refer to the appearance of the composition; for example, for an aqueous based suspension depot composition it may refer to the homogeneity of the suspension being maintained over the storage period referred to.

Physical stability relates to maintenance of the active agent in the same physical form. When the long-acting injectable depot pharmaceutical composition is an aqueous suspension and the active agent is suspended as a crystalline solid, then the physical form (as determined by XRPD) of the suspended solid does not change during the storage period referred to.

Chemical stability relates to low levels of impurities being formed over the storage period referred to. Typically, these are impurities related to the active agent and may be measured by suitable techniques such as HPLC or LC-MS.

Release of the active agent from the compositions of the invention can be determined by methods known in the art. For example, release rates may be determined using in-vitro dissolution tests, which mimic an aqueous physiological-type environment. The term “aqueous physiological-type environment” as used herein refers to the body of a warm blooded animal, particularly human. In particular embodiments, the compositions of the invention are designed for sub-cutaneous or intra-muscular administration and the term “aqueous physiological-type environment” for such compositions refers to the sub-cutaneous or intra-muscular environments of such a body. These conditions may be simulated in vitro by placing a composition in an aqueous dissolution medium, optionally buffered to a physiological pH, at a temperature of from 35 to 40° C. The amount of active released over a given time period may be determined by sampling the dissolution medium and measuring the concentration of the active using a suitable analytical method, for example HPLC.

Release of the active from the compositions of the invention can also be determined by in-vivo methods known in the art. For example, release in vivo can be tested by measuring plasma concentrations at predetermined time periods and thereby obtaining a plasma concentration versus time profile for the compound of interest.

Other tests may also be used to determine the amount of release of the active agent in vivo. Animals (e.g., mice, rats, dogs etc.) may be used as models to investigate release characteristics. For example, for a subcutaneous or intramuscular composition, animals can receive the composition under investigation and after specified periods of time, the animals can be sacrificed and the subcutaneous or intramuscular composition can be extracted/retrieved and analysed. By determining the content of the active agent remaining in the composition at specified periods of time, the amount and extent of release can be calculated.

Advantageously, compositions of the invention are able to maintain a therapeutically effective plasma concentration of the drug throughout a prolonged period.

### Pharmacokinetics

The present invention provides long-acting injectable depot pharmaceutical compositions that provide certain advantages over prior art compositions in terms of providing steady and continuous plasma helicase-primase inhibitor levels for prolonged periods, such that viral shedding and viral reactivation may be reduced or even eliminated.

In an embodiment, the long-acting injectable depot pharmaceutical composition according to the present invention, after administration to a subject in need of treatment thereof, maintains in the subject a geometric mean plasma concentration of the helicase-primase inhibitor of at least 25 ng/mL throughout at least a 14 day period.

In an embodiment, the long-acting injectable depot pharmaceutical composition according to the present invention comprises a helicase-primase inhibitor, or a or a pharmaceutically acceptable salt thereof, which produces a plasma concentration of the helicase-primase inhibitor in the subject after administration of at least 25 ng/mL (such as at least 50 ng/mL, or at least 100 ng/mL) for at least 80% (such as at least 85%, at least 90%, at least 95%, at least 98%, at least 99%, or substantially all) of the dosing interval. Conveniently, the dosing interval is at least 10 days (such as at least 14 days, at least 21 days, at least 28 days, at least 56 days, at least 72 days, at least 96 days, at least 120 days, at least 180 days, at least 240 days, or at least 300 days). In an embodiment, the dosing interval is at least 14 days or at least 28 days. Conveniently, the dosing interval is at least 56 days, at least at least 96 days, at least 120 days, at least 180 days, at least 240 days, or at least 300 days.

In an embodiment, the long-acting injectable depot pharmaceutical composition according to the present invention comprises pritelivir free base and produces a plasma concentration of pritelivir in the subject after administration of at least 25 ng/mL (such as at least 50 ng/mL, or at least 100 ng/mL) for at least 80% (such as at least 85%, at least 90%, at least 95%, at least 98%, at least 99%, or substantially all) of the dosing interval. Conveniently, the dosing interval is at least 10 days (such as at least 14 days, at least 21 days, at least 28 days, at least 56 days, at least 72 days, at least 96 days, at least 120 days, at least 180 days, at least 240 days, or at least 300 days). In an embodiment, the dosing interval is at least 14 days or at least 28 days. Conveniently, the dosing interval is at least 56 days, at least at least 96 days, at least 120 days, at least 180 days, at least 240 days, or at least 300 days.

In an embodiment, the long-acting injectable depot pharmaceutical composition according to the present invention comprises Compound 1 free form and produces a plasma concentration of Compound 1 in the subject after administration of at least 25 ng/mL (such as at least 50 ng/mL, or at least 100 ng/mL) for at least 80% (such as at least 85%, at least 90%, at least 95%, at least 98%, at least 99%, or substantially all) of the dosing interval. Conveniently, the dosing interval is at least 10 days (such as at least 14 days, at least 21 days, at least 28 days, at least 56 days, at least 72 days, at least 96 days, at least 120 days, at least 180 days, at least 240 days, or at least 300 days). In an embodiment, the dosing interval is at least 14 days or at least 28 days. Conveniently, the dosing interval is at least 56 days, at least at least 96 days, at least 120 days, at least 180 days, at least 240 days, or at least 300 days.

The helicase-primase inhibitor is typically subject to plasma protein binding. Dependent on the extent of the plasma protein binding, the free fraction (unbound active agent) may be low relative to the protein bound fraction. For example, pritelivir is typically subject to 97-98% protein binding, so the free fraction may only be 2-3% of the total plasma concentration. In an embodiment, the plasma concentrations referred to above refer to the unbound plasma concentrations. Therefore, in an embodiment, the long-acting injectable depot pharmaceutical composition according to the present invention, after administration to a subject in need of treatment thereof, maintains in the subject a geometric mean unbound plasma concentration of the helicase-primase inhibitor (such as pritelivir) of at least 25 ng/mL throughout at least a 14 day period. In an embodiment, the long-acting injectable depot pharmaceutical composition according to the present invention, after administration to a subject in need of treatment thereof, maintains in the subject a geometric mean total (unbound and protein bound) plasma concentration of the helicase-primase inhibitor (such as pritelivir) of at least 1,500 ng/mL (such as at least 2,000 ng/mL, or at least 2,500 ng/mL) throughout at least a 14 day period.

In an embodiment, the long-acting injectable depot pharmaceutical composition according to the present invention, after administration to a subject in need of treatment thereof, maintains in the subject a geometric mean total (unbound and protein bound) plasma concentration of Compound 1 of at least 500 ng/mL (such as at least 1000 ng/mL, at least 1100 ng/mL, at least 1,500 ng/mL, at least 2,000 ng/mL, or at least 2,500 ng/mL) throughout most if not all of at least a 14 day period. Conveniently, the long-acting injectable depot pharmaceutical composition according to the present invention, after administration to a subject in need of treatment thereof, maintains in the subject a geometric mean total (unbound and protein bound) plasma concentration of Compound 1 of between 500 ng/mL and 1700 ng/mL (such as between 900 ng/mL and 1,300 ng/mL or between 900 ng/mL and 1,200 ng/mL) throughout most if not all of at least a 14 day period.

### Particular Compositions of the Invention

In one embodiment, the present invention provides a long-acting injectable depot pharmaceutical composition comprising:
a) a helicase-primase inhibitor; and
b) one or more pharmaceutically acceptable excipients;
   wherein the helicase-primase inhibitor has a human biological terminal half-life of 10 hours or more; and wherein the helicase-primase inhibitor has a solubility in water or an aqueous solvent system (measured at room temperature) of less than 100 μg/ml. Conveniently, the long-acting injectable depot pharmaceutical composition continuously releases the helicase-primase inhibitor at a rate resulting in therapeutic plasma concentrations of the helicase-primase inhibitor for a period of at least 1 month after subcutaneous or intramuscular administration. Conveniently, the long-acting injectable depot pharmaceutical composition comprises about 1 wt. % to about 80 wt. % of the helicase-primase inhibitor. Conveniently, the long-acting injectable depot pharmaceutical composition comprises about 10 wt. % to about 40 wt. % of the helicase-primase inhibitor. Conveniently, the long-acting injectable depot pharmaceutical composition comprises 300 to 900 mg of the helicase-primase inhibitor and the long-acting injectable depot pharmaceutical composition is administered in an injection volume of about 0.1 to about 3 mL. Conveniently, the long-acting injectable depot pharmaceutical composition comprises an aqueous based suspension, or a solution in organic solvent based formulation. Conveniently, the aqueous based suspension is a microsuspension or nanosuspension. Conveniently, the long-acting injectable depot pharmaceutical composition does not comprise a rate-controlling agent, such as a biodegradable polymer, or contains less than 20% or less than 15, 10, 5, 3, 2 or 1% by weight of the total composition of a rate-controlling agent.

In one embodiment, the present invention provides a long-acting injectable depot pharmaceutical composition comprising:
a) pritelivir free base form (conveniently a hydrate, such as a hemihydrate, sesquihydrate or dihydrate); and
b) one or more pharmaceutically acceptable excipients;
   wherein the long-acting injectable depot pharmaceutical composition continuously releases pritelivir at a rate resulting in therapeutic plasma concentrations of the helicase-primase inhibitor for a period of at least 1 month after subcutaneous or intramuscular administration.

In one embodiment, the present invention provides a long-acting injectable depot pharmaceutical composition comprising:
a) pritelivir free base form (conveniently a dihydrate or sesquihydrate); and
b) one or more pharmaceutically acceptable excipients;
   wherein the long-acting injectable depot pharmaceutical composition continuously releases pritelivir at a rate resulting in therapeutic plasma concentrations of the helicase-primase inhibitor for a period of at least 1 month after subcutaneous or intramuscular administration.

In one embodiment, the present invention provides a long-acting injectable depot pharmaceutical composition comprising:
a) pritelivir free base form (conveniently a hemihydrate, dihydrate or sesquihydrate, such as a sesquihydrate); and
b) one or more pharmaceutically acceptable excipients;
   wherein the long-acting injectable depot pharmaceutical composition comprises 300 to 900 mg of pritelivir and the long-acting injectable depot pharmaceutical composition is administered in an injection volume of about 0.1 to about 3 mL. Conveniently, the long-acting injectable depot pharmaceutical composition continuously releases pritelivir at a rate resulting in therapeutic plasma concentrations of pritelivir for a period of at least 3 months (preferably at least 6 months) after subcutaneous or intramuscular administration. Conveniently, the long-acting injectable depot pharmaceutical composition comprises a solution in organic solvent based formulation comprising pritelivir and wherein pritelivir is dissolved in an organic solvent selected from N-methyl pyrrolidone (NMP), dimethylacetamide (DMA), dimethyl sulfoxide (DMSO), benzyl alcohol, benzyl benzoate and mixtures thereof, preferably N-methyl pyrrolidone (NMP), dimethylacetamide (DMA), and dimethyl sulfoxide (DMSO). The composition optionally further comprises an aqueous media (such as aqueous buffer or saline) and/or a surfactant (such as a polyethylene glycol fatty ester, e.g. PEG-15 hydroxystearate). Conveniently, the long-acting injectable depot pharmaceutical composition comprises an aqueous based suspension. Conveniently, the aqueous based suspension is a nanosuspension.

Conveniently, the long-acting injectable depot pharmaceutical composition does not comprise a rate-controlling agent, such as a biodegradable polymer, or contains less than 20% or less than 15, 10, 5, 3, 2 or 1% by weight of the total composition of a rate-controlling agent. Conveniently, the pritelivir free base form is a sesquihydrate.

In one embodiment, the present invention provides a long-acting injectable depot pharmaceutical composition comprising:
c) pritelivir free base form (conveniently a dihydrate or sesquihydrate); and
d) one or more pharmaceutically acceptable excipients;
   wherein the long-acting injectable depot pharmaceutical composition comprises 300 to 900 mg of the helicase-primase inhibitor, the long-acting injectable depot pharmaceutical composition is administered in an injection volume of about 0.1 to about 2 mL, and wherein the long-acting injectable depot pharmaceutical composition comprises an aqueous based suspension (such as an aqueous based microsuspension). Conveniently, the pritelivir free base form is a sesquihydrate.

In one embodiment, the present invention provides a long-acting injectable depot pharmaceutical composition comprising:
a) Compound 1 free form (conveniently the anhydrous Form A or Form C); and
b) one or more pharmaceutically acceptable excipients;
   wherein the long-acting injectable depot pharmaceutical composition continuously releases Compound 1 at a rate resulting in therapeutic plasma concentrations of Compound 1 for a period of at least 1 month after subcutaneous or intramuscular administration. Conveniently, the long-acting injectable depot pharmaceutical composition is administered by intramuscular injection.

In one embodiment, the present invention provides a long-acting injectable depot pharmaceutical composition comprising:
a) Compound 1 free form (conveniently the anhydrous Form A or Form C); and
b) one or more pharmaceutically acceptable excipients;
   wherein the long-acting injectable depot pharmaceutical composition comprises 300 to 900 mg of Compound 1 and the long-acting injectable depot pharmaceutical composition is administered in an injection volume of about 0.1 to about 3 mL. Conveniently, the long-acting injectable depot pharmaceutical composition continuously releases Compound 1 at a rate resulting in therapeutic plasma concentrations of Compound 1 for a period of at least 3 months (preferably at least 6 months) after subcutaneous or intramuscular administration. Conveniently, the long-acting injectable depot pharmaceutical composition comprises a solution in organic solvent based formulation comprising Compound 1 and wherein Compound 1 is dissolved in an organic solvent selected from N-methyl pyrrolidone (NMP), dimethylacetamide (DMA), dimethyl sulfoxide (DMSO), benzyl alcohol, benzyl benzoate and mixtures thereof, preferably N-methyl pyrrolidone (NMP), dimethylacetamide (DMA), and dimethyl sulfoxide (DMSO). The composition optionally further comprises an aqueous media (such as aqueous buffer or saline) and/or a surfactant (such as a polyethylene glycol fatty ester, e.g. PEG-15 hydroxystearate). Conveniently, the long-acting injectable depot pharmaceutical composition comprises an aqueous based suspension. Conveniently, the aqueous based suspension is a nanosuspension.

Conveniently, the long-acting injectable depot pharmaceutical composition does not comprise a rate-controlling agent, such as a biodegradable polymer, or contains less than 20% or less than 15, 10, 5, 3, 2 or 1% by weight of the total composition of a rate-controlling agent. Conveniently, the Compound 1 form is Form C.

In one embodiment, the present invention provides a long-acting injectable depot pharmaceutical composition comprising:
a) Compound 1 free form (conveniently the anhydrous Form A or Form C); and
b) one or more pharmaceutically acceptable excipients;
   wherein the long-acting injectable depot pharmaceutical composition comprises 300 to 900 mg of Compound 1, the long-acting injectable depot pharmaceutical composition is administered in an injection volume of about 0.1 to about 2 mL, and wherein the long-acting injectable depot pharmaceutical composition comprises an aqueous based suspension, preferably an aqueous based nanosuspension. Conveniently, the Compound 1 form is Form C.

In one embodiment, the present invention provides a long-acting injectable depot pharmaceutical composition comprising:
a) Compound 1 free form as anhydrous Form C; and
b) one or more pharmaceutically acceptable excipients;
   wherein the long-acting injectable depot pharmaceutical composition:
i. comprises 300 to 900 mg of Compound 1;
ii. comprises an aqueous based suspension;
iii. has an injection volume of about 0.1 to about 2 mL; and
iv. continuously releases Compound 1 at a rate resulting in therapeutic plasma concentrations of Compound 1 for a period of at least 6 months after subcutaneous administration.

In one embodiment, the present invention provides a long-acting injectable depot pharmaceutical composition comprising:
a) Compound 1 free form as anhydrous Form C; and
b) one or more pharmaceutically acceptable excipients;
   wherein the long-acting injectable depot pharmaceutical composition:
i. comprises 300 to 900 mg of Compound 1;
ii. comprises an aqueous based suspension;
iii. has an injection volume of about 0.1 to about 2 mL; and
iv. continuously releases Compound 1 at a rate resulting in therapeutic plasma concentrations of Compound 1 for a period of at least 6 months after intramuscular administration.

Suitably the above long-acting injectable depot pharmaceutical compositions comprising Compound 1 in an aqueous based suspension, may also further comprise one or more of an isotonic agent (such as sodium chloride or mannitol), a suspending agent (such as polyvinylpyrrolidone or a polysorbate, e.g. polysorbate 80) and a surfactant (such as a poloxamer, e.g. poloxamer 188). Conveniently, the aqueous based suspension is a microsuspension with a D10 of 0.3 to 2 μm, a D50 of 2 to 4 μm, and/or a D90 of 4 to 13 μm. Conveniently, the aqueous based suspension is a nanosuspension with a D10 of 1 to 100 nm, a D50 of 1 to 150 nm, and/or a D90 of 1 to 200 nm.

In one embodiment, the present invention provides a long-acting injectable depot pharmaceutical composition comprising:
a) Compound 1; and
b) one or more pharmaceutically acceptable excipients;
   wherein the long-acting injectable depot pharmaceutical composition:
i. comprises 300 to 900 mg of Compound 1;
ii. comprises a solution of Compound 1 in NMP;
iii. has an injection volume of about 0.1 to about 2 mL; and
iv. continuously releases Compound 1 at a rate resulting in therapeutic plasma concentrations of Compound 1 for a period of at least 6 months after subcutaneous administration.

Suitably the above long-acting injectable depot pharmaceutical composition comprising Compound 1 in an NMP solution, may also further comprise one or more of an aqueous media (such as aqueous buffer or saline) and a surfactant (such as a polyethylene glycol fatty ester, e.g. PEG-15 hydroxystearate). Conveniently, the composition comprises Compound 1 at concentration of 100-400 mg/mL and 5-95% v/v of NMP.

### Preparation of the Compositions

In a second aspect, the present invention provides a method for forming a pharmaceutical composition according to the first aspect.

In an embodiment, the pharmaceutical composition of the present invention is an aqueous based suspension depot. In an embodiment, a process for the preparation of the aqueous based suspension depot comprises the steps of:
a) incorporating the helicase-primase inhibitor, or a pharmaceutically acceptable salt thereof, in a water based vehicle, wherein the water-based vehicle optionally comprises a suspending agent (e.g. polysorbate 80 or poloxamer 188) and optionally an isotonic agent (e.g. sodium chloride); and
b) mixing the helicase-primase inhibitor, or a pharmaceutically acceptable salt thereof (e.g. by stirring and/or vortexing and/or sonication) to provide a homogenous suspension suitable for subcutaneous or intramuscular administration.

In an embodiment, the pharmaceutical composition of the present invention is an aqueous based microsuspension depot. In an embodiment, a process for the preparation of the aqueous based microsuspension depot comprises the steps of:
a) reducing the particle size of the helicase-primase inhibitor, or a pharmaceutically acceptable salt thereof, drug substance, e.g. by wet or dry milling, to provide micro-sized particles (e.g. Dv90<10 μm);
b) incorporating the micro-sized particles of the helicase-primase inhibitor, or a pharmaceutically acceptable salt thereof, in a water based vehicle, wherein the water-based vehicle optionally comprises a suspending agent (e.g. polysorbate 80 or poloxamer 188) and optionally an isotonic agent (e.g. sodium chloride) and optionally an viscosity modifier (e.g. hydroxypropylmethyl cellulose); and
c) mixing the micro-sized particles of the helicase-primase inhibitor, or a pharmaceutically acceptable salt thereof, in the water based vehicle (e.g. by stirring and/or vortexing and/or sonication) to provide a homogenous suspension suitable for subcutaneous or intramuscular administration.

In an embodiment, the milling conducted in step a) is jet milling.

In an embodiment, the pharmaceutical composition of the present invention is an aqueous based nanosuspension depot. In an embodiment, a process for the preparation of the aqueous based nanosuspension depot comprises the steps of:
a) reducing the particle size of the helicase-primase inhibitor, or a pharmaceutically acceptable salt thereof, drug substance, e.g. by wet milling, to provide nano-sized particles (e.g. Dv90<500 nm);
b) incorporating nano-sized particles of the helicase-primase inhibitor, or a pharmaceutically acceptable salt thereof, in a water based vehicle, wherein the water-based vehicle optionally comprises a suspending agent (e.g. polysorbate 80 or poloxamer 188) and optionally an isotonic agent (e.g. mannitol) and optionally an isotonic agent (e.g. sodium carboxymethyl cellulose); and
c) mixing the nano-sized particles of the helicase-primase inhibitor, or a pharmaceutically acceptable salt thereof, in the water based vehicle (e.g. by stirring and/or vortexing and/or sonication) to provide a homogenous nanosuspension suitable for subcutaneous or intramuscular administration.

In an embodiment, the milling conducted in step a) is wet milling at 700 rpm for at least 300 minutes to reduce particle size to nanosized particles, e.g. Dv90<500 nm.

In an embodiment, reducing the particle size of the helicase-primase inhibitor, or a pharmaceutically acceptable salt thereof, drug substance to provide nano-sized particles (e.g. Dv90<500 nm) for the nanosuspension is carried out in a step-wise fashion. For example, particles of helicase-primase inhibitor, or a pharmaceutically acceptable salt thereof, drug substance are firstly reduced to micro-sized material, e.g. by high pressure homogenizer until the size was around 1 μm. The method then comprises a step to mill this material, e.g. wet milling using zirconium oxide milling beads, e.g. at 700 rpm for 500 minutes, to reduce particle size to nanosized particles (Dv90<500 nm).

### Therapeutic Uses and Applications

The present invention provides a method for treating a herpes virus (conveniently a HSV) infection in a subject in need thereof, the method comprising administering a therapeutically effective amount of a helicase-primase inhibitor, or a pharmaceutically acceptable salt thereof, to the subject wherein the helicase-primase inhibitor or a pharmaceutically acceptable salt thereof, is administered via the injectable route of administration. Conveniently, the helicase-primase inhibitor or a pharmaceutically acceptable salt thereof, is administered by subcutaneous or intramuscular injection.

The present invention provides a method for treating a herpes virus (conveniently a HSV) infection in a subject in need thereof, the method comprising administering a therapeutically effective amount of a helicase-primase inhibitor, or a pharmaceutically acceptable salt thereof, to the subject wherein the helicase-primase inhibitor or a pharmaceutically acceptable salt thereof, is formulated as a pharmaceutical composition according to the first aspect of the invention.

The present invention also provides a pharmaceutical composition according to the first aspect of the invention comprising a helicase-primase inhibitor or a pharmaceutically acceptable salt thereof for use as a medicament.

The present invention also provides a pharmaceutical composition according to the first aspect of the invention comprising a helicase-primase inhibitor or a pharmaceutically acceptable salt thereof for use in the treatment of a herpes virus (conveniently a HSV) infection in a subject in need thereof.

The present invention also provides the use of a pharmaceutical composition according to the first aspect of the invention comprising a helicase-primase inhibitor or a pharmaceutically acceptable salt thereof for the manufacture of a medicament for the treatment of a herpes virus (conveniently a HSV) infection in a subject in need thereof.

In a particular embodiment, the present invention provides a method for inhibiting HSV replication in a subject in need thereof, the method comprising administering a therapeutically effective amount of a helicase-primase inhibitor, or a pharmaceutically acceptable salt thereof, to the subject wherein the helicase-primase inhibitor or a pharmaceutically acceptable salt thereof, is formulated as a pharmaceutical composition according to the first aspect of the invention.

In one embodiment, the present invention also provides a method for reducing the likelihood or severity of symptoms of a HSV infection in a subject in need thereof, the method comprising administering a therapeutically effective amount of a helicase-primase inhibitor, or a pharmaceutically acceptable salt thereof, to the subject wherein the helicase-primase inhibitor or a pharmaceutically acceptable salt thereof, is formulated as a pharmaceutical composition according to the first aspect of the invention.

In one embodiment, the present invention provides a method for inhibiting the development or progression of a disease or disorder caused by, or associated with, HSV infection, in a subject in need thereof, the method comprising administering a therapeutically effective amount of a helicase-primase inhibitor, or a pharmaceutically acceptable salt thereof, to the subject wherein the helicase-primase inhibitor or a pharmaceutically acceptable salt thereof, is formulated as a pharmaceutical composition according to the first aspect of the invention.

In one embodiment, the present invention provides a method for suppressing recurrence of HSV symptoms or outbreaks in a subject in need thereof, the method comprising administering a therapeutically effective amount of a helicase-primase inhibitor, or a pharmaceutically acceptable salt thereof, to the subject wherein the helicase-primase inhibitor or a pharmaceutically acceptable salt thereof, is formulated as a pharmaceutical composition according to the first aspect of the invention.

In an embodiment, the present invention provides a pharmaceutical composition according to the first aspect of the invention comprising a helicase-primase inhibitor or a pharmaceutically acceptable salt thereof for use in suppressing the recurrence of HSV symptoms or outbreaks in a subject in need thereof.

In an embodiment, the present invention provides the use of a pharmaceutical composition according to the first aspect of the invention comprising a helicase-primase inhibitor or a pharmaceutically acceptable salt thereof for the manufacture of a medicament for the suppression of the recurrence of HSV symptoms or outbreaks in a subject in need thereof.

In one embodiment, the present invention provides a method for treating or preventing a disease or disorder caused by, or associated with, HSV infection, in a subject in need thereof, the method comprising administering a therapeutically effective amount of a helicase-primase inhibitor, or a pharmaceutically acceptable salt thereof, to the subject wherein the helicase-primase inhibitor or a pharmaceutically acceptable salt thereof, is formulated as a pharmaceutical composition according to the first aspect of the invention. In a particular embodiment, the disease or disorder caused by, or associated with, HSV infection, is selected from herpes labialis (e.g., oro-labial cold sores or Whitlow's), Herpes genitalis, HSV-related keratitis, HSV-related encephalitis, pneumonia, herpes gladiatorum, primary HSV gingivostomatitis, Mollaret's meningitis, and Bell's palsy.

In a particular embodiment, the disease or disorder caused by, or associated with, HSV infection, is selected from herpes labialis (oro-labial cold sores or Whitlow's) or genital herpes. In one embodiment, the disease or disorder is recurrent herpes labialis or recurrent genital herpes. Individuals with a history of multiple recurrences of herpes labialis or recurrent genital herpes, e.g. HSV which recurs six times or more annually, may be regarded as having recurrent HSV.

In one embodiment, the herpes virus being treated is HSV2. In a further embodiment, the herpes virus being treated is HSV2 and the subject in need of the treatment has HSV2 recurrent genital herpes.

In one embodiment, the herpes virus being treated is HSV1. In yet a further embodiment, both herpes virus HSV1 and HSV2 are being treated.

In one embodiment, the herpes virus being treated is resistant to nucleosidic antiviral therapy. In one embodiment, the nucleosidic antiviral therapy is selected from the group consisting of acyclovir, penciclovir, famciclovir, ganciclovir and valacyclovir.

In one embodiment, the herpes virus infection being treated is resistant to nucleosidic antiviral therapy, e.g., acyclovir-resistant mucocutaneous HSV infection. In a further embodiment, the HSV infection being treated is a mucocutaneous HSV infection resistant to therapy with antiviral therapy with nucleoside analogues, such as acyclovir, penciclovir, famciclovir, ganciclovir or valacyclovir.

In a particular embodiment, the subject in need of the methods disclosed herein, is immunocompromised. The subject may be immunocompromised due to conditions including HIV infection, cancer, hematopoietic cell or solid organ transplantation, chronic glucocorticoid use or a genetic immunodeficiency.

In a particular embodiment, the subject in need of the methods disclosed herein, is a neonate or an infant.

In a particular embodiment, the subject is a herpes-positive patient.

In a particular embodiment, the subject in need of the methods disclosed herein, has acyclovir-resistant mucocutaneous HSV infection. This subject may have been diagnosed with this condition on the basis of clinical failure, e.g., no improvement after oral or iv doses for at least 7 days with approved doses of acyclovir.

In a particular embodiment, the subject in need of the methods disclosed herein, has a primary genital HSV-related herpes infection. In one embodiment, the subject in need of the methods disclosed herein, has severe or progressive genital HSV-related herpes infection.

In a particular embodiment, the pharmaceutical compositions according to the first aspect of the invention can reduce recurrence of HSV infections (i.e., provide a suppressive therapy) causing diseases or disorders, such as herpes labialis or genital herpes. In one embodiment, the reduction in the number of recurrences of lesions over a period of one year can be reduced by 20, 30, 40, 50, 75, 90 or 95% or more. Conveniently, the rate of lesions over one year can be reduced by 90% or more.

In a particular embodiment, the pharmaceutical compositions according to the first aspect of the invention can reduce the duration of recurrent episodes of HSV infection, e.g., by one or more days, e.g., at least 2, 3, 4, 5, 14, 21 or 28 days.

In a particular embodiment, the pharmaceutical compositions according to the first aspect of the invention can reduce time to healing of lesions (e.g., time to full recovery of lesions) and duration of symptoms resulting from HSV infections in diseases or disorders, such as herpes labialis or genital herpes. The time to lesion healing may be defined as complete epithelization of mucocutaneous HSV lesion(s) within the treatment period and no appearance of new lesions, e.g., as assessed by a physician.

In one embodiment, the pharmaceutical compositions according to the first aspect of the invention can reduce pain or pain intensity (for example, at a lesion site) caused as a consequence of HSV infections in diseases or disorders, such as herpes labialis or genital herpes.

In a particular embodiment, the pharmaceutical compositions according to the first aspect of the invention can reduce viral shedding or reduce the rate of viral shedding in individuals with frequently recurring HSV, e.g., genital HSV2. For example, a within-subject genital HSV mucocutaneous shedding rate can be measured by taking swabs of skin and mucosa and for HSV detection, e.g. by analysing samples for HSV DNA with a real-time, quantitative, fluorescent polymerase-chain-reaction (PCR) assay. The frequency of HSV2 detection (the viral shedding rate) can be defined as the number of days with a genital swab that was positive for HSV divided by the total number of days on which genital swabs were obtained. Reduction in the HSV shedding rate among subjects receiving the compositions of the invention relative to the shedding rate among subjects receiving placebo or other treatments can be compared. The quantity of HSV in positive swabs and the frequency of genital lesions and shedding episodes can also be monitored.

In a particular embodiment, the present invention provides a method for reducing (or substantially suppressing or eliminating) break-through HSV shedding in a subject in need thereof, the method comprising administering a therapeutically effective amount of a helicase-primase inhibitor, or a pharmaceutically acceptable salt thereof, to the subject wherein the helicase-primase inhibitor or a pharmaceutically acceptable salt thereof, is formulated as a pharmaceutical composition according to the first aspect of the invention.

In an embodiment, the present invention provides a pharmaceutical composition according to the first aspect of the invention comprising a helicase-primase inhibitor or a pharmaceutically acceptable salt thereof for use in reducing (or substantially suppressing or eliminating) break-through HSV shedding in a subject in need thereof.

In an embodiment, the present invention provides the use of a pharmaceutical composition according to the first aspect of the invention comprising a helicase-primase inhibitor or a pharmaceutically acceptable salt thereof for the manufacture of a medicament for the reduction (or substantial suppression or elimination) of break-through HSV shedding in a subject in need thereof.

In a particular embodiment, the present invention provides a method for preventing transmission of an infectious disease caused by HSV, the method comprising administering a therapeutically effective amount of a helicase-primase inhibitor, or a pharmaceutically acceptable salt thereof, to the subject wherein the helicase-primase inhibitor or a pharmaceutically acceptable salt thereof, is formulated as a pharmaceutical composition according to the first aspect of the invention.

In a particular embodiment, the present invention provides a method for reducing side effects observed when a helicase-primase inhibitor, or a pharmaceutically acceptable salt thereof, is administered via an oral route of administration to a subject with an infectious disease caused by HSV, the method comprising administering a therapeutically effective amount of a helicase-primase inhibitor, or a pharmaceutically acceptable salt thereof, to the subject wherein the helicase-primase inhibitor or a pharmaceutically acceptable salt thereof, is formulated as a pharmaceutical composition according to the first aspect of the invention.

Pharmaceutical compositions according to the present invention are injectable depot compositions and are therefore to be dosed by intramuscular or subcutaneous administration. In a convenient embodiment, the pharmaceutical compositions according to the present invention are administered by subcutaneous administration. In a convenient embodiment, the pharmaceutical compositions according to the present invention are administered by intramuscular administration.

For use in accordance with this aspect, the appropriate dosage is expected to vary depending on, for example, the nature and severity of the infection to be treated and is within the purview of the treating physician. Usually, an indicated administration dose may be in the range between about 0.1 to about 1000 μg/kg body weight. In some cases, the administration dose of the compound may be less than 400 μg/kg body weight. In other cases, the administration dose may be less than 200 μg/kg body weight. In yet other cases, the administration dose may be in the range between about 0.1 to about 100 μg/kg body weight. In an embodiment, the therapeutically effective amount of the helicase-primase inhibitor or a pharmaceutically acceptable salt thereof, is about 5 mg to about 900 mg, such as about 100 mg to about 600 mg.

Advantageously, the applicants have found that the prolonged release provided by the compositions of the invention and the resultant steady plasma levels kept at a therapeutically effective plasma concentration over a prolonged period after administration allow the possibility of using lower than expected doses.

The dose may be conveniently administered twice a month or less, once a month or less, once every two, three, four, five or six months or less. Conveniently, the dose may be conveniently administered once a month, once every two months or once every three months.

In a convenient embodiment, the unit dosage form of the pharmaceutical composition is a subcutaneous injection. In a convenient embodiment, the unit dosage form of the pharmaceutical composition comprises about 5 mg to about 900 mg (such as about 100 mg to about 900 mg) of the helicase-primase inhibitor, or a pharmaceutically acceptable salt thereof. In a most convenient embodiment, the unit dosage form of the pharmaceutical composition is a subcutaneous injection comprising about 100 mg to about 600 mg of the helicase-primase inhibitor, or a pharmaceutically acceptable salt thereof.

In a convenient embodiment, the unit dosage form of the pharmaceutical composition comprises about 100 mg to about 1200 mg (such as about 100 mg to about 900 mg) of Compound 1, or a pharmaceutically acceptable salt thereof. In a most convenient embodiment, the unit dosage form of the pharmaceutical composition is a subcutaneous injection comprising about 100 mg to about 900 mg of Compound 1, or a pharmaceutically acceptable salt thereof. In a yet more convenient embodiment, the unit dosage form of the pharmaceutical composition is a subcutaneous injection comprising about 200 mg to about 900 mg, such as about 200 mg to about 600 mg, about 300 mg to about 900 mg, or about 300 mg to about 600 mg of Compound 1, or a pharmaceutically acceptable salt thereof.

In one embodiment, the present invention provides a method for treating a herpes virus (conveniently a HSV) infection in a subject in need thereof, the method comprising administering a therapeutically effective amount of a helicase-primase inhibitor, or a pharmaceutically acceptable salt thereof, to the subject wherein the helicase-primase inhibitor or a pharmaceutically acceptable salt thereof, is formulated as a pharmaceutical composition according to the first aspect of the invention (for example, certain long-acting injectable depot pharmaceutical compositions comprising a solution in organic solvent or an aqueous microsuspension or nanosuspension), wherein no loading dose, such as an oral administration loading dose, is required prior to administration of long-acting injectable depot pharmaceutical composition according to the first aspect of the invention.

### Combinations

Pharmaceutical compositions of the present invention may be administered alone as a sole therapy or can be administered in addition with one or more other substances and or treatments. Such conjoint treatment may be achieved by way of simultaneous, sequential or separate administration of the individual components of the treatment.

Also contemplated herein are methods that include administering a second active agent. For example, in addition to being infected with HSV, a subject or patient can further have HSV infection-related co-morbidities, i.e., diseases and other adverse health conditions associated with, exacerbated by, or precipitated by being infected with HSV. Contemplated herein are also disclosed pharmaceutical compositions in combination with at least one other agent that has previously been shown to treat these HSV-infection-related conditions. Such conjoint treatment may be achieved independently (by way of simultaneous, sequential or separate administration of the individual components of the treatment) and/or via pharmaceutical compositions of the present invention that include a second active agent.

Therefore, provided herein is a method for treating or preventing HSV infection in a subject in need thereof, the method comprising administering a therapeutically effective amount of a helicase-primase inhibitor, or a pharmaceutically acceptable salt thereof, to the subject, wherein the helicase-primase inhibitor or a pharmaceutically acceptable salt thereof is formulated as a pharmaceutical composition according to the first aspect of the invention, and co-administering to the subject a therapeutically effective amount of an additional therapeutic agent.

In an embodiment, the additional therapeutic agent is selected from one or more of the following agents:
i. nucleoside polymerase inhibitors, such as acyclovir, valacyclovir, famciclovir, penciclovir and ganciclovir;
ii. pyrophosphate polymerase inhibitors, such as foscarnet;
iii. saturated aliphatic alcohols, such as docosanol;
iv. agents such as idoxuridine, trifluridine and vidarabine;
v. a corticosteroid; and
vi. other helicase-primase inhibitors, such as amenamevir.

In some cases, a disclosed pharmaceutical composition according to the first aspect of the invention may be administered as part of a combination therapy in conjunction with one or more antivirals, including nucleoside analogues such as acyclovir, foscarnet, ganciclovir or penciciovir or the respective prodrugs valaciclovir or famciclovir.

In some embodiments, the first and second amounts together comprise a pharmaceutically effective amount. The first amount, the second amount, or both may be the same, more, or less than effective amounts of each compound administered as monotherapies. Therapeutically effective amounts of a disclosed compound and antiviral may be co-administered to the subject, i.e., administered to the subject simultaneously or separately, in any given order and by the same or different routes of administration. In some instances, it may be advantageous to initiate administration of Compound 1 first, for example one or more days or weeks prior to initiation of administration of the antiviral. Moreover, additional drugs may be given in conjunction with the above combination therapy.

### Kits

In one embodiment, the pharmaceutical compositions and methods described herein provide kits for the treatment of disorders, such as the one described herein. These kits comprise a pharmaceutical composition described herein in a container and, optionally, instructions teaching the use of the kit according to the various methods and approaches described herein. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

The pharmaceutical compositions of the invention may be utilized for diagnostics and as research tools.

Besides being useful for human treatment, pharmaceutical compositions of the invention, may be useful for veterinary treatment of companion animals, exotic animals and farm animals, including mammals, rodents, and the like. Conveniently, such animals include horses, dogs and cats.

The invention is illustrated below by the following non-limiting examples.

### EXAMPLES

The following abbreviations are used within this specification:

| | |
|---|---|
| ACN: | Acetonitrile |
| DCM: | Dichloromethane |
| DMF: | Dimethylformamide |
| DMSO: | Dimethyl sulfoxide |
| DSC: | Differential Scanning Calorimetry |
| DVS: | Dynamic Vapor Sorption |
| EDCI: | 1-Ethyl-3-(3-dimethylaminopropyl)carbodiimide |
| eq: | Equivalents |
| EtOAc: | Ethyl acetate |
| hr: | Hours |
| HPLC: | High Performance Liquid Chromatography |
| K₂CO₃: | Potassium carbonate |
| K₃PO₄: | Tripotassium phosphate |
| KF: | Karl Fischer titration |
| MeOH: | Methanol |
| min: | Minutes |
| NaCMC: | Sodium carboxymethyl celulose |
| Na₂SO₄: | Sodium sulfate |
| NMP: | N-Methyl Pyrrolidone |
| NMT: | not more than |
| PLM: | Polarized light microscopy |
| PVP K30: | Polyvinylpyrrolidone K30 grade (Povidone K30) |
| RH: | Relative Humidity |
| RT: | Room Temperature (~22° C.) |
| TBAB: | Tetrabutylammonium bromide |
| TGA: | Thermogravimetric analysis |
| THF: | Tetrahydrofuran |
| TLC: | Thin Layer Chromatography |
| Vol: | Volume |
| XRPD: | X-Ray Powder Diffraction |

### Materials and Methods

### Particle Size Distribution by Light Diffraction

The particle size distribution of the suspensions prepared below in Examples 1 and 10 were determined by laser diffraction using a NANOTRAC FLEX particle size analyser (Microtrac Instruments). Water was used as the medium. Sample was added to keep the laser obscuration level at 0.1-10%. A measurement time of 20 seconds was used, and background measurements were collected using water for 10 seconds. Dv₁₀, Dv₅₀ and Dv₉₀ diameters were determined and are reported below.

The particle size distribution of the suspensions prepared in Examples 14 and 17 were determined using a Mastersizer 3000 particle size analyser, Malvern, UK. The suspension was freeze dried and the freeze-dried powder was used to prepare a 0.1% suspension in water for testing. Water was used as dispersant. The suspension was added until the final obscuration range was 3-10%. Each sample was measured a total of 3 times. Dv₁₀, Dv₅₀ and Dv₉₀ diameters were determined and are reported below.

### Scanning Electron Microscopy

Scanning electron microscopy (SEM) images of the suspensions prepared below in Examples 14 and 17 were taken using an Oxford X-MAS scanning electron microscope (Zeiss-Sigma-HD, Germany). Before measurement, the suspension was diluted with distilled water (4×) and sonicated for about 5 mins. The sample was then dipped on conductive adhesive and air dried at room temperature. Loose powder was removed by use of a rubber suction bulb. The sample was then sprayed with gold for testing.

### LC-MS Analysis of Plasma Samples

The LC-MS methodology and conditions used to analyse pritelivir in plasma samples from the PK studies described in Examples 1 to 5 are provided in Table 1.

TABLE 1
LC-MS Parameters for pritelivir quantification in plasma samples

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 (2.1 × 50 mm, 1.7 μm) | | |
| Flow rate | 0.60 mL/min | | |
| Column | 60° C. | | |
| Temperature | | | |
| Sample diluent | MeOH:H2O 50:50 (v:v) | | |
| Mobile Phase A | Water/0.025% Formic acid/1M Ammonium Acetate | | |
| Mobile Phase B | Methanol/5 mM Ammonium Acetate | | |
| Program Type | Gradient | | |
| | Time | % Mobile | % Mobile |
| Gradient Program | (min) | Phase A | Phase B |
| | 0.0 | 90 | 0 |
| | 0.20 | 90 | 10 |
| | 1.20 | 10 | 90 |
| | 1.80 | 10 | 90 |
| | 1.81 | 90 | 10 |
| | 2.20 | stop | stop |

| | |
|---|---|
| Sample Temperature | Room Temperature |
| Injection Volume | 0.5 μL |
| MS Detection Mode | Multiple reaction monitoring (MRM) |
| Retention time | 1.49 min (pritelivir) |

The LC-MS methodology and conditions used to analyse pritelivir in dog plasma samples from the PK study described in Example 14 are provided in Table 2.

TABLE 2
LC-MS Parameters for pritelivir quantification
in dog plasma samples

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 (2.1 × 50 mm, 1.7 μm) | | |
| Flow rate | 0.60 mL/min | | |
| Column | 60° C. | | |
| Temperature | | | |
| Sample diluent | MeOH:H2O 50:50 (v:v) | | |
| Mobile Phase A | Water/0.025% Formic acid/1M Ammonium Acetate | | |
| Mobile Phase B | Methanol/0.025% Formic acid/1M Ammonium | | |
| | Acetate | | |
| Program Type | Gradient | | |
| | Time | % Mobile | % Mobile |
| Gradient Program | (min) | Phase A | Phase B |
| | 0.0 | 90 | 0 |
| | 0.20 | 90 | 10 |
| | 1.20 | 10 | 90 |
| | 1.80 | 10 | 90 |
| | 1.81 | 90 | 10 |
| | 2.20 | stop | stop |

| | |
|---|---|
| Sample Temperature | Room Temperature |
| Injection Volume | 0.2 μL |
| MS Detection Mode | Multiple reaction monitoring (MRM) |
| Retention time | 1.49 min (pritelivir) |

The LC-MS methodology and conditions used to analyse the helicase-primase inhibitor Compound 1 in plasma samples from the sub-cutaneous injection PK studies (Examples 7 and 10-12) are described in Table 3 and Table 4.

TABLE 3
LC-MS Parameters for Compound 1 quantification
in rat plasma samples

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 (2.1 × 50 mm, 1.7 μm) | | |
| Flow rate | 0.60 mL/min | | |
| Column | 60° C. | | |
| Temperature | | | |
| Sample diluent | MeOH:H2O 50:50 (v:v) | | |
| Mobile Phase A | Water/0.025% Formic acid/1M Ammonium Acetate | | |
| Mobile Phase B | Methanol/5 mM Ammonium Acetate | | |
| Program Type | Gradient | | |
| | Time | % Mobile | % Mobile |
| Gradient Program | (min) | Phase A | Phase B |
| | 0.0 | 95 | 5 |
| | 0.30 | 95 | 5 |
| | 1.50 | 10 | 90 |
| | 2.00 | 10 | 90 |
| | 2.01 | 95 | 5 |
| | 2.60 | stop | stop |

| | |
|---|---|
| Sample Temperature | Room Temperature |
| Injection Volume | 2.0 μL |
| MS Detection Mode | Multiple reaction monitoring (MRM) |
| Retention time | 1.69 min (Compound 1) |

TABLE 4
LC-MS Parameters for Compound 1 quantification
in dog and monkey plasma samples

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 (2.1 × 50 mm, 1.7 μm) | | |
| Flow rate | 0.60 mL/min | | |
| Column | 60° C. | | |
| Temperature | | | |
| Sample diluent | MeOH:H2O 50:50 (v:v) | | |
| Mobile Phase A | Water/0.025% Formic acid/1M Ammonium Acetate | | |
| Mobile Phase B | Methanol/5 mM Ammonium Acetate | | |
| Program Type | Gradient | | |
| | Time | % Mobile | % Mobile |
| Gradient Program | (min) | Phase A | Phase B |
| | 0.0 | 90 | 10 |
| | 0.20 | 90 | 10 |
| | 1.00 | 10 | 90 |
| | 1.80 | 10 | 90 |
| | 1.81 | 90 | 10 |
| | 2.20 | stop | stop |

| | |
|---|---|
| Sample Temperature | Room Temperature |
| Injection Volume | 1.0 μL |
| MS Detection Mode | Multiple reaction monitoring (MRM) |
| Retention time | 1.39 min (Compound 1) |

The LC-MS methodology and conditions used to analyse Compound 1 in plasma samples from the intra-venous injection PK studies (Example 13) are described in Tables 5 to 7.

TABLE 5
LC-MS Parameters for Compound 1 quantification
in rat plasma samples following intra-venous
administration in study described in Example 13

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 (2.1 × 50 mm, 1.7 μm) | | |
| Flow rate | 0.60 mL/min | | |
| Column | 60° C. | | |
| Temperature | | | |
| Sample diluent | MeOH:H2O 50:50 (v:v) | | |
| Mobile Phase A | Water/0.025% Formic acid/1 mM Ammonium Acetate | | |
| Mobile Phase B | Methanol/5 mM Ammonium Acetate | | |
| Program Type | Gradient | | |
| | Time | % Mobile | % Mobile |
| | (min) | Phase A | Phase B |
| Gradient Program | 0.0 | 90 | 10 |
| | 0.20 | 90 | 10 |
| | 1.20 | 10 | 90 |
| | 1.80 | 10 | 90 |
| | 1.81 | 90 | 10 |
| | 2.20 | stop | stop |

| | |
|---|---|
| Sample | Room Temperature |
| Temperature | |
| Injection Volume | 2.0 μL |
| MS Detection | Multiple reaction monitoring (MRM) |
| Mode | |
| Retention time | 1.43 min (Compound 1) |

TABLE 6
LC-MS Parameters for Compound 1 quantification
in dog plasma samples following intra-venous
administration in study described in Example 13

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 (2.1 × 50 mm, 1.7 μm) | | |
| Flow rate | 0.60 mL/min | | |
| Column | 60° C. | | |
| Temperature | | | |
| Sample diluent | MeOH:H2O 50:50 (v:v) | | |
| Mobile Phase A | Water/0.025% Formic acid/1 mM Ammonium Acetate | | |
| Mobile Phase B | Methanol/5 mM Ammonium Acetate | | |
| Program Type | Gradient | | |
| | Time | % Mobile | % Mobile |
| | (min) | Phase A | Phase B |
| Gradient Program | 0.0 | 90 | 10 |
| | 0.20 | 98 | 2 |
| | 1.20 | 98 | 2 |
| | 1.80 | 10 | 90 |
| | 1.81 | 90 | 10 |
| | 2.20 | stop | stop |

| | |
|---|---|
| Sample | Room Temperature |
| Temperature | |
| Injection Volume | 1.0 μL |
| MS Detection | Multiple reaction monitoring (MRM) |
| Mode | |
| Retention time | 1.48 min (Compound 1) |

TABLE 7
LC-MS Parameters for Compound 1 quantification
in monkey plasma samples following intra-venous
administration in study described in Example 13

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 (2.1 × 50 mm, 1.7 μm) | | |
| Flow rate | 0.60 mL/min | | |
| Column | 60° C. | | |
| Temperature | | | |
| Sample diluent | MeOH:H2O 50:50 (v:v) | | |
| Mobile Phase A | Water/0.025% Formic acid/1 mM Ammonium Acetate | | |
| Mobile Phase B | Methanol/5 mM Ammonium Acetate | | |
| Program Type | Gradient | | |
| | Time | % Mobile | % Mobile |
| | (min) | Phase A | Phase B |
| Gradient Program | 0.0 | 95 | 5 |
| | 0.30 | 95 | 5 |
| | 1.50 | 10 | 90 |
| | 2.00 | 10 | 90 |
| | 2.01 | 95 | 5 |
| | 2.60 | stop | stop |

| | |
|---|---|
| Sample | Room Temperature |
| Temperature | |
| Injection Volume | 1.0 μL |
| MS Detection | Multiple reaction monitoring (MRM) |
| Mode | |
| Retention time | 1.69 min (Compound 1) |

The LC-MS methodology and conditions used to analyse the helicase-primase inhibitor Compound 1 in rat plasma samples from the sub-cutaneous injection PK study described in Example 15 are provided in Table 8.

TABLE 8
LC-MS Parameters for Compound 1 quantification
in rat plasma samples

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 (2.1 × 50 mm, 1.7 μm) | | |
| Flow rate | 0.60 mL/min | | |
| Column | 50° C. | | |
| Temperature | | | |
| Sample diluent | MeOH:H₂O 50:50 (v:v) | | |
| Mobile Phase A | Water/0.025% Formic acid/1M Ammonium Acetate | | |
| Mobile Phase B | Methanol/0.025% Formic acid/1M Ammonium | | |
| | Acetate | | |
| Program Type | Gradient | | |
| | Time | % Mobile | % Mobile |
| | (min) | Phase A | Phase B |
| Gradient Program | 0.0 | 80 | 20 |
| | 0.20 | 80 | 20 |
| | 1.70 | 5 | 95 |
| | 2.00 | 5 | 95 |
| | 2.01 | 80 | 20 |
| | 2.50 | stop | stop |

| | |
|---|---|
| Sample | Room Temperature |
| Temperature | |
| Injection Volume | 1.0 μL |
| MS Detection | Multiple reaction monitoring (MRM) |
| Mode | |
| Retention time | 1.83 min (Compound 1) |

The LC-MS methodology and conditions used to analyse the helicase-primase inhibitor Compound 1 in dog plasma samples from the dog PK study described in Example 16 are given in Table 9.

TABLE 9
LC-MS Parameters for Compound 1 quantification
in dog plasma samples

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 (2.1 × 50 mm, 1.7 μm) | | |
| Flow rate | 0.60 mL/min | | |
| Column | 50° C. | | |
| Temperature | | | |
| Sample diluent | MeOH:H₂O 50:50 (v:v) | | |
| Mobile Phase A | Water/0.025% Formic acid/1M Ammonium Acetate | | |
| Mobile Phase B | Methanol/0.025% Formic acid/1M Ammonium | | |
| | Acetate | | |
| Program Type | Gradient | | |
| | Time | % Mobile | % Mobile |
| | (min) | Phase A | Phase B |
| Gradient Program | 0.0 | 90 | 10 |
| | 0.20 | 90 | 10 |
| | 0.40 | 40 | 60 |
| | 1.40 | 10 | 90 |
| | 1.80 | 10 | 90 |
| | 1.81 | 90 | 10 |
| | 2.20 | stop | stop |

| | |
|---|---|
| Sample | Room Temperature |
| Temperature | |
| Injection Volume | 1.0 μL |
| MS Detection | Multiple reaction monitoring (MRM) |
| Mode | |
| Retention time | 1.44 min (Compound 1) |

The LC-MS methodology and conditions used to analyse Compound 1 in rat plasma samples from the PK study described in Example 17 are given in Table 10.

TABLE 10
LC-MS Parameters for Compound 1 quantification
in rat plasma samples

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 (2.1 × 50 mm, 1.7 μm) | | |
| Flow rate | 0.60 mL/min | | |
| Column | 50° C. | | |
| Temperature | | | |
| Sample diluent | MeOH:H₂O 50:50 (v:v) | | |
| Mobile Phase A | Water/0.025% Formic acid/1M Ammonium Acetate | | |
| Mobile Phase B | Methanol/0.025% Formic acid/1M Ammonium | | |
| | Acetate | | |
| Program Type | Gradient | | |
| | Time | % Mobile | % Mobile |
| | (min) | Phase A | Phase B |
| Gradient Program | 0.0 | 90 | 10 |
| | 0.20 | 90 | 10 |
| | 1.20 | 10 | 90 |
| | 1.80 | 10 | 90 |
| | 1.81 | 90 | 10 |
| | 2.20 | stop | stop |

| | |
|---|---|
| Sample | Room Temperature |
| Temperature | |
| Injection Volume | 1 μL |
| MS Detection | Multiple reaction monitoring (MRM) |
| Mode | |
| Retention time | 1.33 min (Compound 1) |

### X-Ray Powder Diffraction (XRPD) Analysis

Instrument: Rigaku Smartlab SE
X-Ray Wavelength: Cu, Kα, Kα1 (Å): 1.540598, Kα2(Å): 1.544426
Kα2: Kα1 intensity ratio:0.50
X-Ray Tube Setting: 40 kV, 15 mA, Scan Mode 10D
Scan Range (2 Theta): 3°-40°, Step Size (2 Theta): 0.02° with Scan Speed (2 Theta) 10°/min

### Pritelivir Free Base (Dihydrate)

Pritelivir (100 mg) was dissolved in 88:12 THF/water (1 mL) at −60° C. This solution was cooled and then water (4 mL) was added. The resultant suspension was filtered, the solid was collected and dried.

The crystalline pritelivir free base obtained according to this procedure was analysed as described below and was used in Examples 1 to 6 described herein.

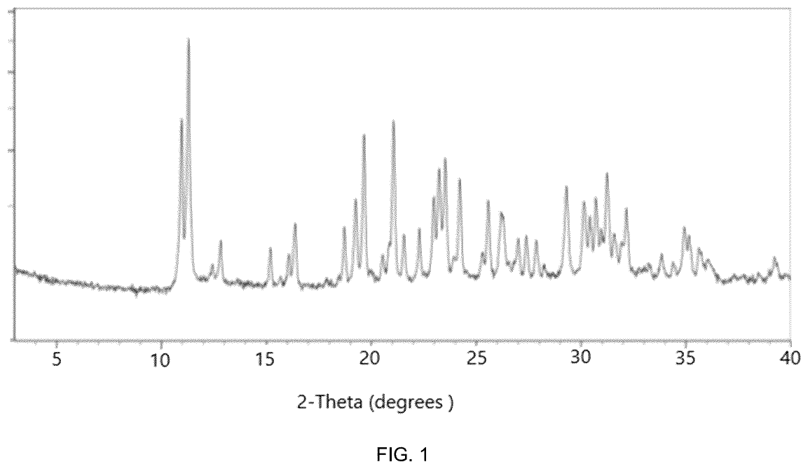

Pritelivir free base was analysed by TGA and XRPD. By TGA the material lost 7.9% weight as water between 30° C. and 90° C., corresponding to a dihydrate. The XRPD diffractogram is shown in FIG. 1 and the peak positions are listed in the table below. Characteristic peaks include those at 11.0, 11.3 and 21.1°2θ.

Peak Position °2θ

11.0
11.3
12.4
12.8
15.2
15.7
16.1
16.4
18.5
18.7
19.3
19.7
20.5
21.1
21.6
22.3
23.0
23.2
23.5
23.9
24.2
24.5
25.3
25.6
26.2
27.0
27.4
27.9
29.3
30.1
30.4
30.7
31.0
31.2
31.6
31.9
32.1
33.2
33.8
34.4
34.9
35.2
35.6
36.1
39.0
39.2

### Pritelivir Free Base (Sesquihydrate)

2-(4-(pyridin-2-yl)phenyl)acetic acid (38.40 g, 80%, 180.074 mmol, 1.00 eq) was added to DMF (385 mL). HOBT (17.38 g, 128.624 mmol, 0.71eq) was added at 20° C., and then stirred for 10 minutes. 2-chloro-4-methylthiazole-5-sulfonamide (26.66 g, 128.624 mmol, 0.71eq) and EDCl (27.12 g, 141.486 mmol, 0.79 eq) were added. The mixture was stirred for 36 hours at 20° C. under N₂. The mixture was added into water (780 mL) and stirred for 30 minutes. The solid was collected by filtration. The filter cake was washed with water twice (2×100 mL) and then dried under reduced pressure at 50° C. for 12 hours to give pritelivir as a white solid (yield 78.4%).

The crystalline pritelivir free base obtained according to this procedure was analysed as described below and was used in Example 14.

Pritelivir free base was analysed by TGA and XRPD. By TGA, the material lost 5.6% weight between 35 and 101° C. The characterization data suggests that this is a sesquihydrate of pritelivir (1.5 water molecules per 1 molecule of pritelivir). The XRPD diffractogram is shown in FIG. 16 and the peak positions are listed in the table below. Characteristic peaks include those at 12.1, 14.6 and 18.3°2θ.

Peak Position °2θ

7.2
8.7
10.3
12.1
14.6
15.0
15.4
16.3
18.3
19.9
20.4
20.7
20.9
21.5
22.1
22.4
22.6
23.7
23.9
24.1
24.5
24.8
25.6
26.1
26.9
27.6
28.0
28.7
29.4
29.6
30.5
30.8
31.9
32.3
33.2
33.8
34.4
34.9
35.4
35.8
36.3
36.5
36.9
37.8
38.8
39.5
39.8

### Compound 1 Free Base (Anhydrous Form A)

Crude Compound 1 prepared according to Example 8 (36.5 g) was stirred in DMSO (300 mL, 8.2 relative volumes) at 15-27° C. for 20 to 30 minutes to form a solution. Water (1100 mL, 30.1 relative volumes) was added slowly over 30 mins and the mixture stirred at 15-27° C. for 1-2 hours. Additional water (1820 mL, 49.9 relative volumes) was added slowly over 60 mins and the mixture stirred at 15-27° C. for 12 hours. The mixture was then filtered, washed with water (3×365 mL, 3×10 volumes) and the cake dried at 45 to 55° C. to give 26.4 g (72% yield) of Compound 1 as an off white solid.

The crystalline Compound 1 free base Form A obtained according to this procedure was analysed as described below and was used in Examples 7 and 10-12.

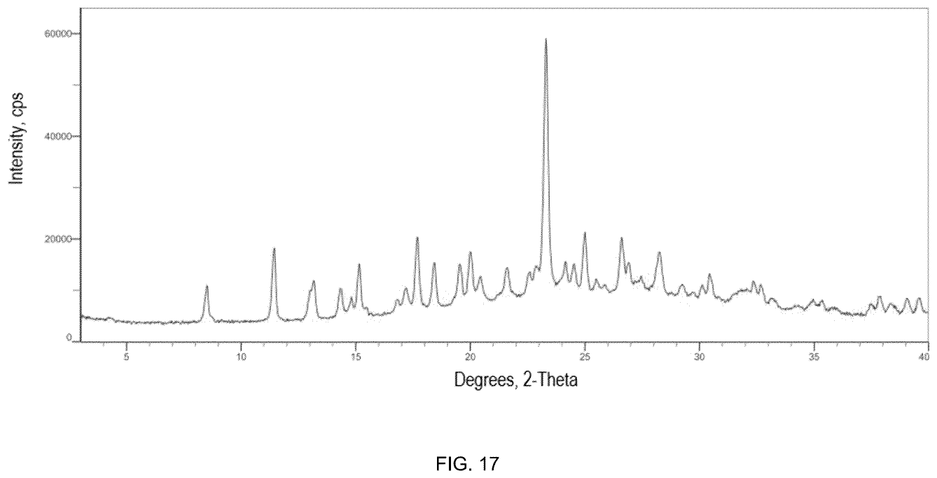

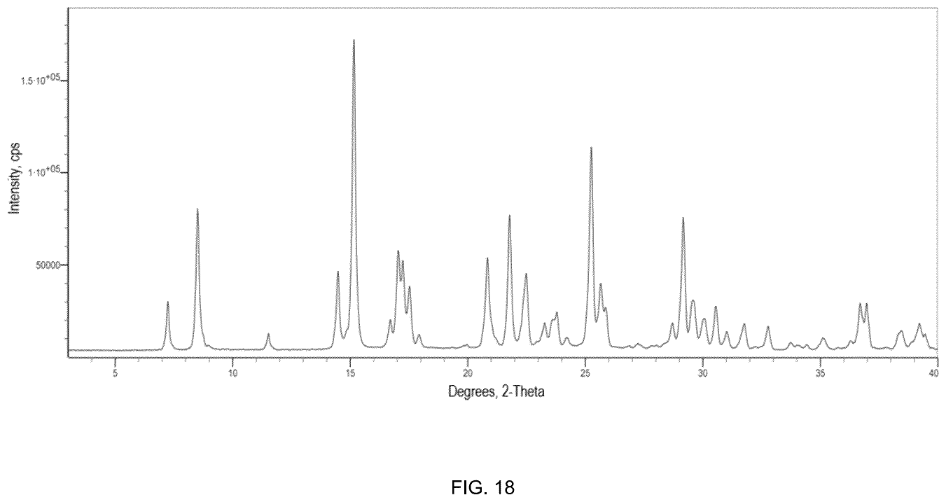

The crystalline form obtained had an XRPD diffractogram as shown in FIG. 17 and was designated as Form A. Peak positions present in the XRPD diffractogram acquired for Form A are listed in the table below.

| Peak position in °2θ | Relative Intensity (%) |
|---|---|
| 8.4 | 21 |
| 11.4 | 63 |
| 13.1 | 56 |
| 14.3 | 12 |
| 15.1 | 53 |
| 17.1 | 12 |
| 17.6 | 100 |
| 18.4 | 15 |
| 19.5 | 14 |
| 19.9 | 36 |
| 20.3 | 17 |
| 21.5 | 9 |
| 23.2 | 93 |
| 24.9 | 16 |
| 26.5 | 18 |
| 26.8 | 23 |
| 27.4 | 4 |
| 28.1 | 20 |
| 31.7 | 9 |
| 32.2 | 9 |

Further solid state characterization of Form A was performed using DSC and TGA and Form A was determined to be an anhydrous crystalline form of Compound 1.

### Compound 1 Free Base (Anhydrous Form C)

Crude Compound 1 prepared according to Example 8 (4.0 kg) was stirred in DMSO (12 L, 3 relative volumes) at 75-85° C. in a first reactor to form a solution. The solution was then filtered through a 0.2 μm filter into a second pre-warmed (75 to 85° C.) reactor, the first reactor being subsequently rinsed with DMSO (2 L, 0.5 rel. vol.) and the rinse being transferred to the second reactor. The contents of the second reactor were stirred at 75-85° C. in for 30-60 minutes and then cooled at a cooling rate of 8-12° C. per hour to give a batch temperature of 50-55° C. The mixture was stirred at 50-55° C. for 1-2 hr. Ethanol (4 L, 1 rel. vol.) was then added slowly over a period of 60-90 minutes whilst maintaining the batch temperature at 50-55° C. The internal batch temperature was increased to 57 to 62° C. and ethanol (52 L, 13 rel. vol.) was added slowly over a period of 7.0-7.5 hr. The contents were stirred at 57 to 62° C. for 1 to 2 hours and then cooled at a cooling rate of 8-12° C. per hour to give a final batch temperature of 18-22° C. The mixture was stirred at 18-22° C. for 8-12 hours and was then filtered, washed with ethanol (8 L, 2 volumes) and the cake dried at 45 to 55° C. for 20-24 hr to give 3.6 kg (90% yield) of Compound 1 as an off white to light yellow solid.

The crystalline Compound 1 free base obtained according to this procedure was analysed as described below and was used in Example 17.

The crystalline form obtained had an XRPD diffractogram as shown in FIG. 18 and was designated as Form C. Peak positions present in the XRPD diffractogram acquired for Form C are presented in the following table:

| Peak position in °2θ | Relative Intensity (%) |
|---|---|
| 7.3 | 15 |
| 8.5 | 44 |
| 14.5 | 23 |
| 15.1 | 100 |
| 17.0 | 29 |
| 17.2 | 25 |
| 17.5 | 19 |
| 20.8 | 28 |
| 21.8 | 41 |
| 22.4 | 11 |
| 22.5 | 20 |
| 25.3 | 66 |
| 25.7 | 19 |
| 25.9 | 12 |
| 29.2 | 42 |
| 29.6 | 16 |
| 30.0 | 9 |
| 30.5 | 14 |
| 36.7 | 16 |
| 37.0 | 16 |

Further solid state characterization of Form C was performed using DSC, TGA and DVS. Form C was determined to be an anhydrous crystalline form of Compound 1.

### Antiviral Assays

The following assay can be used to determine the in-vitro EC₅₀ of a helicase primase inhibitor against HSV-1 and HSV-2.

### Cytopathic Effect (CPE) Inhibition Assay

Cell culture—vero cells were cultured in Dulbecco's Modified Eagle Medium (DMEM) supplemented with 10% fetal bovine serum and 100 units/mL penicillin and streptomycin. The cells were passaged 2-3 times per week to maintain sub-confluent densities.

HSV-1 antiviral assay—vero cells were seeded into 96-well plates at a density of 2.5×10³ cells per well and allowed to attach overnight. Following attachment, the media was replaced with 50 μL of infection medium (DMEM supplemented with 2% fetal bovine serum and 100 units/mL penicillin and streptomycin). A Tecan D300e digital dispenser was then used to add compounds to the culture using an 8-point 3-fold serial dilution format. The DMSO concentration was normalized to 0.5% for all treatments. Following compound addition, 50 μL of infection medium containing 80 TCID₅₀ HSV-1 was added to the cells and incubated at 37° C. for 4 days. After the incubation, the plates were equilibrated to room temperature, the media was removed, and 60 μL of a 1:1 dilution of Cell titer glow and phosphate buffered saline was added to the cells. Following a 5-minute incubation, cell viability was quantified by measuring luminance using a Tecan Infinite M1000 Pro plate reader.

HSV-2 antiviral assay—vero cells were seeded into 96-well plates at a density of 1.0×10⁴ cells per well and allowed to attach overnight. Following attachment, the media was replaced with 50 μL of infection medium (DMEM supplemented with 2% fetal bovine serum and 100 units/mL penicillin and streptomycin). A Tecan D300e digital dispenser was then used to add compounds to the culture using an 8-point 3-fold serial dilution format. The DMSO concentration was normalized to 0.5% for all treatments. Following compound addition, 50 μL of infection medium containing 160 TCID₅₀ HSV-2 G strain was added to the cells and incubated at 37° C. for 5 days. After the incubation, 10 μL/well of WST-8 chromogenic reagent was added and the plates incubated at 37° C. for 3 hours. Following the incubation, cell viability was quantified by measuring the absorbance at 460 nm and 620 nm using a Tecan Infinite M1000 Pro plate reader.

Using this CPE assay, pritelivir had mean EC₅₀ values of 0.047 μM and 0.055 μM against HSV-1 and HSV-2 respectively.

### Example 1: Pritelivir PK Study in Rat

### Formulation Preparation

An aqueous suspension of crystalline pritelivir free base (dihydrate) in a 0.9% NaCl+0.5% polysorbate 80 vehicle at concentrations of 10.0 mg/mL and 50.0 mg/mL was prepared as follows:
1) Vehicle (0.9% w/w NaCl and 0.5% w/w polysorbate 80) was prepared by dissolving NaCl and polysorbate 80 in water.
2) Appropriate amount of crystalline pritelivir free base (dihydrate) was weighed and added to the vehicle.
3) Mixture was vortexed for 1 minute, stirred for 10 minutes and then sonicated for 15 minutes.
4) The final dosing formulation was a homogenous white cloudy suspension.

### Formulation Characterisation

Particle size distribution (PSD) of the suspension was determined as Dv₁₀=1.45 μm, Dv₅₀=4.23 μm and Dv₉₀=10.35 μm.

### Animal Dosing

Male Sprague Dawley (SD) rats with ~300 g body weight (ages 6-8 weeks) were used for the pharmacokinetic study. The animals had free access to food and water. The pritelivir suspensions described above were administered via sub-cutaneous injection to each animal group (n=3) with a dosing volume of 3.33 mL/kg to achieve 33.33 mg/kg and 166.67 mg/kg doses.

At each time point, approximately 200 μL whole blood was collected in a K2EDTA tube via the jugular vein. Blood samples were put on ice and centrifuged at 2000 g for 5 min to obtain a plasma sample within 15 minutes. Plasma samples were stored at approximately −70° C. until analyzed by LC-MS.

### Results

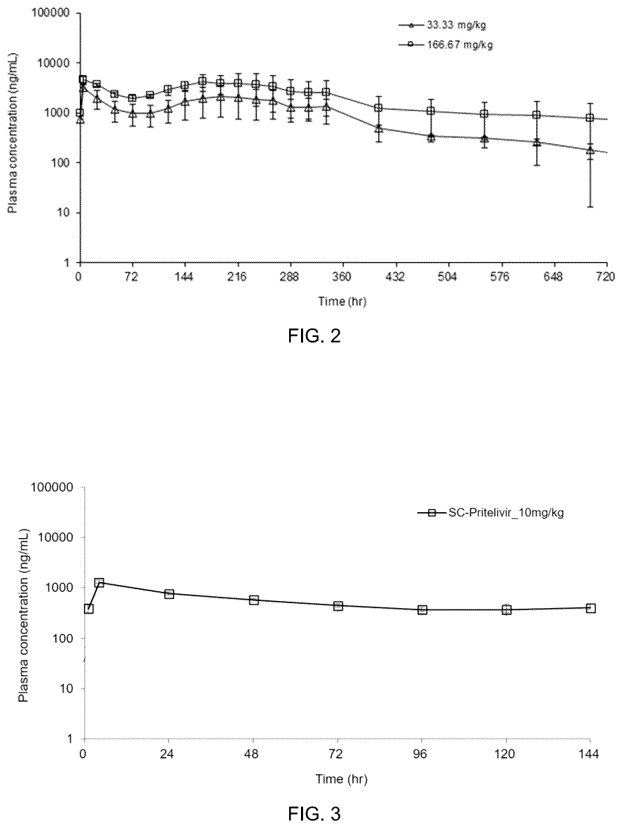

The mean plasma concentration profiles (n=3) after subcutaneous (SC) administration of the pritelivir suspension at two different dosing levels (33.33 mg/kg and 166.67 mg/kg) are shown in FIG. 2. Prolonged exposure out to at least 720 hours (length of the study) was achieved at both dosing levels.

### Example 2: Pritelivir PK Study in Rat at Lower Dose

### Formulation Preparation

An aqueous suspension of crystalline pritelivir free base (dihydrate) in a 0.9% NaCl+0.5% polysorbate 80 vehicle at a concentration of 3.0 mg/mL was prepared as follows:
1) Vehicle (0.9% w/w NaCl and 0.5% w/w polysorbate 80) was prepared by dissolving NaCl and polysorbate 80 in water.
2) Appropriate amount of crystalline pritelivir free base (dihydrate) was weighed and added to the vehicle.
3) Mixture was stirred for 15 minutes, then vortexed for 1 minute and then sonicated for 10 minutes.
4) The final dosing formulation was a homogenous white cloudy suspension.

### Animal Dosing

Male SD rats with 230-250 g body weight, ages 6-8 weeks were used for the rat PK study. The animals had free access to food and water. The prepared suspension was administered via subcutaneous injection to each animal group (n=3) with a dosing volume of 3.33 mL/kg to achieve a 10 mg/kg dose.

At each time point, approximately 200 μL whole blood was collected via the jugular vein into K2EDTA tubes. Blood samples were put on to ice and centrifuged at 2000 g for 5 min to obtain plasma sample within 15 minutes. Plasma samples were stored at approximately −70° C. until analyzed by LC-MS.

### Results

The mean plasma concentration profile (n=3) after subcutaneous (SC) administration of the pritelivir suspension is shown in FIG. 3. Prolonged exposure out to at least 144 hours (length of the study) was achieved at the lower dosing level of 10 mg/kg.

### Example 3: Pritelivir PK Study in Monkey

### Formulation Preparation

An aqueous suspension of crystalline pritelivir free base (dihydrate) in a 0.9% NaCl+0.5% polysorbate 80 vehicle at a concentration of 3.0 mg/mL was prepared as follows:
1) Vehicle (0.9% w/w NaCl and 0.5% w/w polysorbate 80) was prepared by dissolving NaCl and polysorbate 80 in water.
2) Appropriate amount of crystalline pritelivir free base (dihydrate) was weighed and added to the vehicle.
3) Mixture was stirred for 15 minutes, then vortexed for 1 minute and then sonicated for 10 minutes.
4) The final dosing formulation was a homogenous white cloudy suspension.

### Animal Dosing

Male non-naïve Cyno Monkeys with −5 kg body weight was used for the monkey PK study. The animals had free access to food and water. The prepared suspension was administered via subcutaneous injection to each animal group (n=3) with a dosing volume of 3.33 mL/kg to achieve a 10 mg/kg dose.

At each time point, approximately 0.5 mL whole blood was collected via the cephalic vein into K2EDTA tubes. Blood samples were put on to ice and centrifuged at 2000 g for 5 min to obtain plasma sample within 15 minutes. Plasma samples were stored at approximately −70° C. until analyzed by LC-MS.

### Results

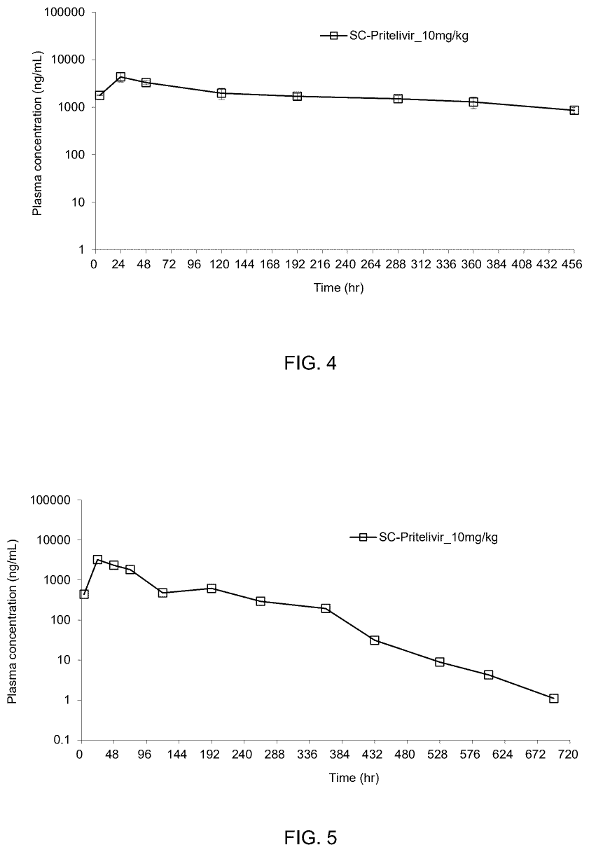

The mean plasma concentration profile (n=3) after subcutaneous (SC) administration of the pritelivir suspension is shown in FIG. 4. Prolonged exposure was achieved out to at least 456 hours (length of the study) at the administered dosing level of 10 mg/kg.

### Example 4: Pritelivir PK Study in Dog

### Formulation Preparation

An aqueous suspension of crystalline pritelivir free base (dihydrate) in a 0.9% NaCl+0.5% poloxamer 188 vehicle at a concentration of 5.0 mg/mL was prepared as follows:
1) Vehicle (0.9% w/w NaCl and 0.5% w/w poloxamer 188) was prepared by dissolving NaCl and poloxamer 188 in water.
2) Appropriate amount of crystalline pritelivir free base (dihydrate) was weighed and added to the vehicle.
3) Mixture was stirred for 15 minutes, then vortexed for 1 minute and then sonicated for 10 minutes.
4) The final dosing formulation was a homogenous white cloudy suspension.

### Animal Dosing

Male non-naïve beagles were used for the dog PK study. The animals had free access to food and water. The prepared suspension was administered via subcutaneous injection to each animal group (n=3) with a dosing volume of 2 mL/kg to achieve a 10 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Results

The mean plasma concentration profile (n=3) after subcutaneous (SC) administration of the pritelivir suspension is shown in FIG. 5. Following an initial drug burst, prolonged exposure was achieved at the administered dosing level.

Summary from PK Studies

In 3 different species, injectable long acting depot formulations containing pritelivir have been found to provide continuous and steady plasma levels of the drug for significantly prolonged periods of time following a single subcutaneous administration. Furthermore, prolonged release has been achieved at different dosing levels.

In addition, there were no adverse reactions observed at the injection sites in the study animals.

### Example 5: Comparative Pritelivir PK Study in Rat Following IV/SC Administration

### Formulation Preparation

A solution of crystalline pritelivir free base (dihydrate) in 5% NMP, 5% Solutol HS15 & 90% saline at a concentration of 0.5 mg/mL was prepared as follows:
1) A stock solution of 10 mg/mL of pritelivir (dihydrate) in NMP was prepared.
2) Equal volumes of the pritelivir stock solution and Solutol HS15 were combined and mixed.
3) The above solution was diluted with normal saline to reach target 0.5 mg/mL pritelivir concentration. The formulation was a clear solution.

### Animal Dosing

Male Sprague Dawley (SD) rats with ~300 g body weight (ages 6-8 weeks) were used for the pharmacokinetic study. The animals had free access to food and water. The pritelivir solution described above was administered via intravenous injection to animals (n=3) with a dosing volume of 2 mL/kg to achieve a 1 mg/kg dose. For subcutaneous injection, the dosing volume of 10 mL/kg was utilized to achieve a 5 mg/kg dose.

At each time point, approximately 200 μL whole blood was collected in a K2EDTA tube via the jugular vein. Blood samples were put on ice and centrifuged at 2000 g for 5 min to obtain a plasma sample within 15 minutes. Plasma samples were stored at approximately −70° C. until analyzed by LC-MS according to the method described above.

### Results

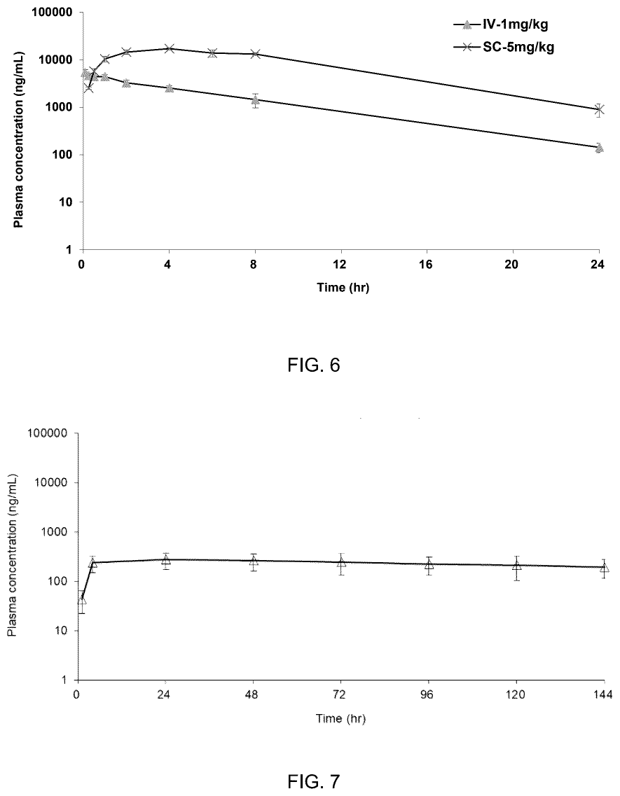

The mean plasma concentration profiles (n=3) after intravenous (IV) at 1 mg/kg and subcutaneous (SC) at 5 mg/kg administration of pritelivir solution can be found in FIG. 6. The calculated F for the SC route is about 117% relative to IV route (Table below).

| | Intravenous (IV) | Subcutaneous (SC) |
|---|---|---|
| Dose (mg/kg) | 1 | 5 |
| AUC(last) (hr*ng/mL) (n = 3) | 31,807 | 185,412 |
| F | 100% | 116.6% |

This rat PK study shows that the SC administration route provides good exposure for pritelivir, with the SC bioavailability surprisingly being comparable to the IV bioavailability.

### Example 6: Pritelivir Suspension (dihydrate) Stability and Solubility Study

### Formulation Preparation

An aqueous suspension of crystalline pritelivir free base (dihydrate) in a 0.9% NaCl+0.5% polysorbate 80 vehicle was prepared as follows:
1) Vehicle (0.9% w/w NaCl and 0.5% w/w polysorbate 80) was prepared by dissolving NaCl and polysorbate 80 in water.
2) Excess amounts of pritelivir (dihydrate) were weighed and added to the vehicle to achieve −3 mg/mL suspension.
3) The suspension was sonicated for 15 minutes, then agitated at 500 rpm at room temperature.
4) At 24 hours, the suspension was filtrated and the filter cake was dried and analyzed by XRPD. The filtrate was analyzed by HPLC to determine solubility.

### HPLC Analysis

The HPLC methodology and conditions used to quantify pritelivir in the filtrate are described in Table 11.

TABLE 11
HPLC Parameters for pritelivir quantification in filtrate

| Parameter | Value | | |
|---|---|---|---|
| Instrument | Agilent 1260 | | |
| Column | ACE ultracore super C18 (100 mm × 4.6 mm × 2.5 | | |
| | μm) (P/N: CORE-25A-1046U) | | |
| Flow rate | 1.0 mL/min | | |
| Column | 20° C. | | |
| Temperature | | | |
| Mobile Phase A | 5 mM diammonium hydrogen phosphate and 5 mM of | | |
| | monoammonium phosphate in water | | |
| Mobile Phase B | Acetonitrile | | |
| | Time | % Mobile | % Mobile |
| | (min) | Phase A | Phase B |
| Gradient Program | Initial | 90 | 10 |
| | 4 | 60 | 40 |
| | 11 | 60 | 40 |
| | 13 | 20 | 80 |
| | 20 | 20 | 80 |
| | 21 | 90 | 10 |
| | 25 | 90 | 10 |

| | |
|---|---|
| Sample | Room Temperature |
| Temperature | |
| Injection Volume | 5 μL |
| Needle wash | Acetonitrile/water (50:50 v/v) |
| Detection Method | UV |
| Detection | 234 nm |
| Wavelength | |
| Retention time | ~4 mins |
| Acquisition time | 25 min |

### Results

| Input | | Insoluble | Solubility |
|---|---|---|---|
| material | Temperature | material | (μg/mL, n = 1) |
| Dihydrate | RT | Dihydrate | 16.5 |

XRPD analysis of the residual solids after the solubility experiment confirmed it as the dihydrate (i.e., no polymorphic transformation occurred during the experiment).

### Example 7: PK Studies with Helicase-Primase Inhibitor, Compound 1

An additional helicase-primase inhibitor (HPI), i.e. Compound 1 (described in Example 8), was also subjected to animal PK testing when formulated as a long-acting injectable depot composition according to the present invention.

Compound 1 was isolated as a crystalline solid and possessed mean in vitro EC₅₀ values of 0.015 μM and 0.013 μM against HSV-1 and HSV-2 respectively in the CPE assay described above.

### PK Study in Rat

### Formulation Preparation

An aqueous suspension of crystalline Compound 1 (Form A) was prepared in a 0.9% NaCl+0.5% polysorbate 80 vehicle at a concentration of 3.0 mg/mL as described in Example 2.

The suspension was then dosed to male SD rats at 10 mg/kg according to the Example 2 animal dosing method.

### Results

The mean plasma concentration profile (n=3) after subcutaneous (SC) administration of the Compound 1 suspension at 10 mg/kg is shown in FIG. 7. Prolonged exposure out to at least 144 hours (length of the study) was achieved at 10 mg/kg for Compound 1 when formulated as the aqueous suspension.

### PK Study in Monkey

### Formulation Preparation

An aqueous suspension of crystalline Compound 1 (Form A) was prepared in a 0.9% NaCl+0.5% polysorbate 80 vehicle at a concentration of 3.0 mg/mL as described in Example 3.

The suspension was then dosed to male cyno monkeys at 10 mg/kg according to the Example 3 animal dosing method.

### Results

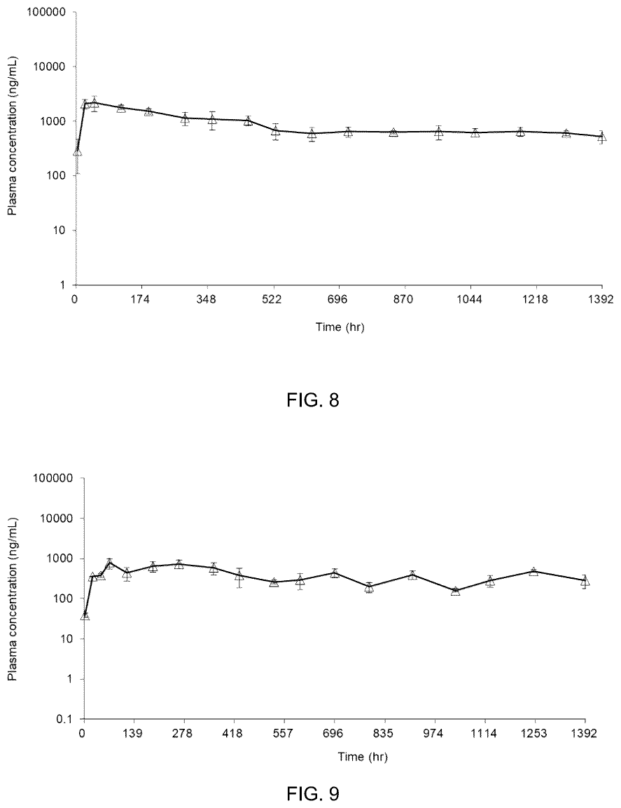

The mean plasma concentration profile (n=3) after subcutaneous (SC) administration of the Compound 1 suspension at 10 mg/kg is shown in FIG. 8. Prolonged exposure out to at least 1392 hours (length of the study) was achieved at 10 mg/kg for Compound 1 when formulated as the aqueous suspension.

### PK Study in Dog

### Formulation Preparation

An aqueous suspension of crystalline Compound 1 (Form A) was prepared in a 0.9% NaCl+0.5% poloxamer 188 vehicle at a concentration of 5.0 mg/mL as described in Example 4.

The suspension was then dosed to male non-naïve beagle dogs at 10 mg/kg according to the Example 4 animal dosing method.

### Results

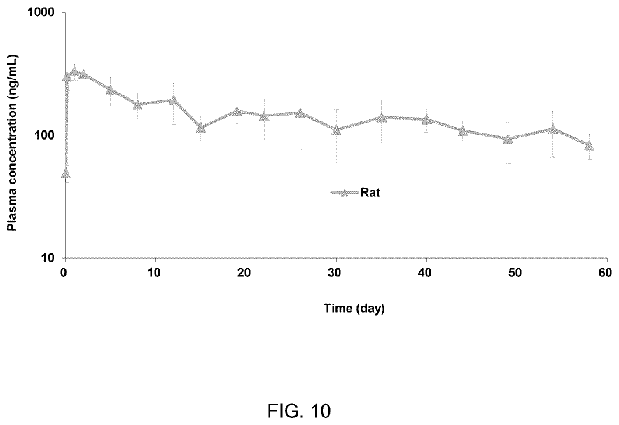

The mean plasma concentration profile (n=3) after subcutaneous (SC) administration of the Compound 1 suspension at 10 mg/kg is shown in FIG. 9. Prolonged exposure out to at least 1392 hours (length of the study) was achieved at 10 mg/kg for Compound 1 when formulated as the aqueous suspension.

### Summary from Example 7 PK Studies with Compound 1

In 3 different species, injectable long acting depot formulations containing Compound 1 have been found to provide continuous and steady plasma levels of the drug for significantly prolonged periods of time following a single subcutaneous administration. Furthermore, prolonged release has been achieved at different dosing levels. In addition, there were no adverse reactions observed at the injection sites in the study animals.

### Example 8: Synthesis of 2-(3-(2′,5′-difluoro-[1,1′-biphenyl]-4-yl)-2-oxotetrahydropyrimidin-1(2H)-yl)-4-methylthiazole-5-sulfonamide (Compound 1)

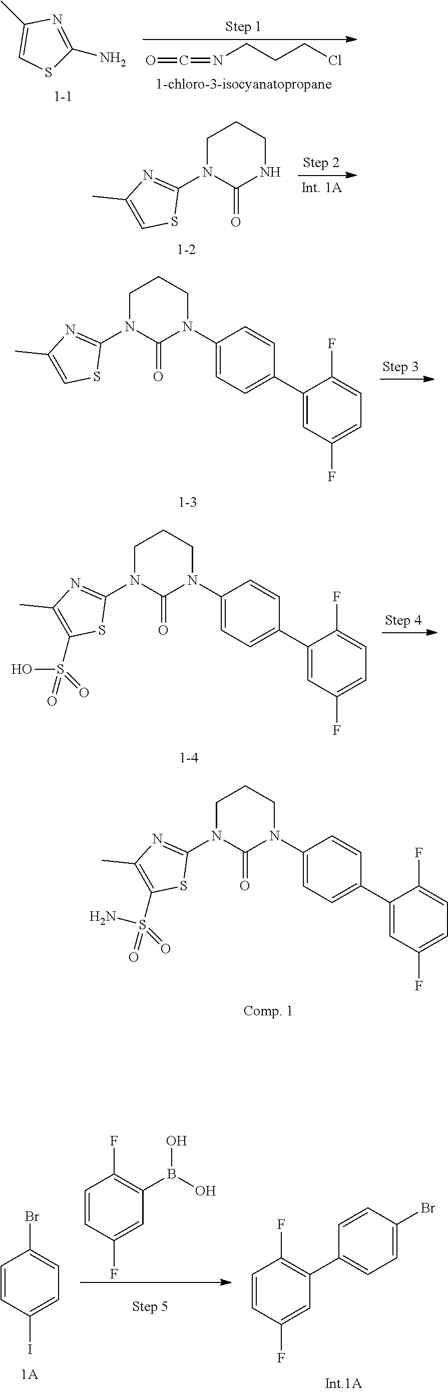

### Synthesis of 1-(4-methylthiazol-2-yl) tetrahydropyrimidin-2(1H)-one (1-2)

A mixture of compound 1-1 (6 g, 52.632 mmol) and 1-chloro-3-isocyanatopropane (6.26 g, 52.632 mmol) in THF (60 mL) was heated at 70° C. for 6 h. To the resulting solution, TBAB (1.7 g, 5.263 mmol) and K₂CO₃ (18.15 g, 131.58 mmol) were added portion wise maintaining the same temperature and stirring continued at 70° C. for 16 h. After completion of the reaction (monitored by TLC), the reaction mixture was diluted with water and extracted with EtOAc. The combined organic layers were dried over anhydrous Na₂SO₄ filtered and concentrated under reduced pressure. The crude compound was purified by CombiFlash chromatography (eluting with 60-70% EtOAc in heptane) to afford the title compound 1-2 (5.1 g, 49.22%) as an off-white solid. TLC: 70% EtOAc/heptane (R_{f}: 0.5). MS calcd. for Chemical Formula: C₈H₁₁N₃OS: 197.06; Found: 198.17 [M+1]⁺. ¹H NMR (400 MHz, DMSO-d₆) δ 7.30 (s, 1H), 6.60 (s, 1H), 3.99 (t, J=5.4 Hz, 2H), 3.20-3.19 (m, 2H), 2.28 (s, 3H), 1.99-1.89 (m, 2H).

### Synthesis of 1-(2′,5′-difluoro-[1,1′-biphenyl]-4-yl)-3-(4-methylthiazol-2-yl) tetrahydropyrimidin-2(1H)-one (1-3)

To a stirred solution of compound 1-2 (5 g, 25.380 mmol) in 1,4-dioxane (100 mL) were added Int. 1A (8.16 g, 30.456 mmol), K₂CO₃ (8.75 g, 63.45 mmol) followed by CuI (0.96 g, 5.076 mmol) and the resulting reaction mixture was purged under nitrogen for 20 min. To this resulting reaction mixture, 1,2-Dimethylethylenediamine (0.9 g, 10.152 mmol) was added under nitrogen atmosphere. The reaction mixture was heated at 120° C. for 24 h in a sealed tube. After completion of the reaction, the reaction mixture was filtered through Celite bed and washed with ethyl acetate. The filtrate was diluted with water and, extracted with EtOAc followed by brine. The combined organic layers were dried over anhydrous Na₂SO₄, filtered and concentrated under reduced pressure. The crude compound obtained was purified by CombiFlash chromatography (eluting with 30-40% EtOAc in heptane) to afford the title compound 1-3 (4.1 g, 41.96%) as an off-white solid. TLC: 50% EtOAc/Heptane (R_{f}: 0.5). MS calcd. for Chemical Formula: C₂₀H₁₇F₂N₃OS: 385.11; Found: 385.90 [M+1]⁺. ¹H NMR (400 MHz, DMSO-d₆) δ 7.61 (d, J=7.8 Hz, 2H), 7.54-7.35 (m, 4H), 7.35-7.21 (m, 1H), 6.70 (s, 1H), 4.17 (t, J=5.6 Hz, 2H), 3.81 (t, J=4.9 Hz, 2H), 2.26 (s, 3H), 2.24-2.21 (m, 2H).

### Synthesis of 2-(3-(2′,5′-difluoro-[1,1′-biphenyl]-4-yl)-2-oxotetrahydropyrimidin-1(2H)-yl)-4-methylthiazole-5-sulfonic acid (1-4)

To a stirred solution of compound 1-3 (4 g, 10.389 mmol) in dry DCM (40 mL) at 0° C. in an inert atmosphere, chlorosulfuric acid (2.07 mL, 31.168 mmol) was added and the resulting reaction mixture was slowly warmed to room temperature and stirred for 12 h. After completion of the reaction, the reaction mixture was concentrated under reduced pressure to dryness. The crude residue obtained was purified by trituration with diethyl ether. The obtained solid was filtered off and dried in vacuo to afford the title compound 1-4 (3.35 g, crude) as an off-white solid. TLC: 100% EtOAc (R_{f}: 0.2). MS calcd. for Chemical Formula: C₂₀H₁₇F₂N₃O₄S₂: 465.06; Found: 466 [M+1]⁺.

### Synthesis of 2-(3-(2′,5′-difluoro-[1,1′-biphenyl]-4-yl)-2-oxotetrahydropyrimidin-1(2H)-yl)-4-methylthiazole-5-sulfonamide (Compound 1)

A stirred solution of compound 1-4 (3.3 g, 7.096 mmol) in POCl₃ (33 mL) was allowed to stir at 90° C. for 5 h. The reaction mixture was concentrated under reduced pressure to dryness. The resulting residue obtained was dissolved in THF (66 mL), and aqueous ammonia (33 mL) was added at −5° C. and stirring continued at room temperature for another 12 h. After completion of the reaction, the reaction mixture was diluted with water and extracted with EtOAc. The combined organic layers were dried over anhydrous Na₂SO₄ filtered and concentrated under reduced pressure. The crude compound was purified by CombiFlash chromatography (eluting with 100% EtOAc) to afford the desired product Compound 1 (1.1 g, 44.64%) as a white solid. ¹H NMR (400 MHz, DMSO-d₆) δ 7.65-7.59 (m, 2H), 7.55 (br s, 2H), 7.53-7.48 (m, 2H), 7.48-7.36 (m, 2H), 7.31-7.25 (m, 1H), 4.17 (t, J=6.1 Hz, 2H), 3.82 (t, J=5.6 Hz, 2H), 2.45 (s, 3H), 2.29-2.18 (m, 2H).

### Synthesis of 4′-bromo-2,5-difluoro-1,1′-biphenyl (Int. 1A)

To a stirred solution of compound 1A (5 g, 17.674 mmol) in 1,4 dioxane: H₂O (50:5 mL) were added (2,5-difluorophenyl) boronic acid (3.07 g, 19.441 mmol) and K₃PO₄ (7.5 g, 35.348 mmol) and the reaction mixture was purged under nitrogen for 10 min. To this resulting solution PdCl₂(dppf) (1.29 g, 1.767 mmol) was added under nitrogen atmosphere. The reaction mixture was heated at 80° C. for 1 h. After completion of the reaction (monitored by TLC), the reaction mixture was cooled to room temperature, filtered through a pad of Celite and washed with ethyl acetate. The filtrate was diluted with water and extracted with EtOAc. The combined organic layers were dried over anhydrous Na₂SO₄, filtered and concentrated under reduced pressure. The crude compound was purified by CombiFlash chromatography (eluting with 100% heptane) to afford the title compound Int. 1A (2.3 g, 48.62%) as an off-white solid. TLC: 100% heptane (R_{f}: 0.5). ¹H NMR (400 MHz, CDCl₃) b 7.58 (d, J=8.3 Hz, 2H), 7.40 (d, J=7.3 Hz, 2H), 7.15-7.06 (m, 2H), 7.05-6.97 (m, 1H).

### Example 9: Biological assay data for 2-(3-(2′,5′-difluoro-[1,1′-biphenyl]-4-yl)-2-oxotetrahydropyrimidin-1(2H)-yl)-4-methylthiazole-5-sulfonamide (Compound 1)

### Cell Culture

Vero cells were cultured in Dulbecco's Modified Eagle Medium (DMEM) supplemented with 10% foetal bovine serum and 100 units/mL penicillin and streptomycin. The cells were passaged 2-3 times per week to maintain sub-confluent densities.

### HSV-1 Antiviral Assay

Vero cells were seeded into 96-well plates at a density of 2.5×10³ cells per well and allowed to attach overnight. Following attachment, the media was replaced with 50 μL of infection medium (DMEM supplemented with 2% foetal bovine serum and 100 units/mL penicillin and streptomycin). A Tecan D300e digital dispenser was then used to add compounds to the culture using an 8-point 3-fold serial dilution format. The DMSO concentration was normalized to 0.5% for all treatments. Following compound addition, 50 μL of infection medium containing 80 TCID₅₀ HSV-1 was added to the cells and incubated at 37° C. for 4 days. After the incubation, the plates were equilibrated to room temperature, the media was removed, and 60 of a 1:1 dilution of Cell titer glow and phosphate buffered saline was added to the cells. Following a 5-minute incubation, cell viability was quantified by measuring luminance using a Tecan Infinite M1000 Pro plate reader.

### HSV-2 Antiviral Assay

Vero cells were seeded into 96-well plates at a density of 1.0×10⁴ cells per well and allowed to attach overnight. Following attachment, the media was replaced with 50 μL of infection medium (DMEM supplemented with 2% foetal bovine serum and 100 units/mL penicillin and streptomycin). A Tecan D300e digital dispenser was then used to add compounds to the culture using an 8-point 3-fold serial dilution format. The DMSO concentration was normalized to 0.5% for all treatments. Following compound addition, 50 μL of infection medium containing 160 TCID₅₀ HSV-2 G strain was added to the cells and incubated at 37° C. for 5 days. After the incubation, 10 μL/well of WST-8 chromogenic reagent was added and the plates incubated at 37° C. for 3 hours. Following the incubation, cell viability was quantified by measuring the absorbance at 460 nm and 620 nm using a Tecan Infinite M1000 Pro plate reader.

In the HSV-1 antiviral assay, Compound 1 had an EC₅₀ of 0.019 M (n=22). In the HSV-2 antiviral assay, Compound 1 had an EC₅₀ of 0.011 M (n=35).

### Example 10: Compound 1 PK Study in Rat

### Formulation Preparation

An aqueous suspension of Compound 1 free form (crystalline Form A) in a 0.9% NaCl+0.5% polysorbate 80 vehicle at a concentration of 3 mg/mL was prepared as follows:
1) Vehicle (0.9% w/w NaCl and 0.5% w/w polysorbate 80) was prepared by dissolving NaCl and polysorbate 80 in water.
2) An appropriate amount of Compound 1 free form was weighed and added to the vehicle.
3) The mixture was vortexed for 1 minute, stirred for 10 minutes and then sonicated for 15 minutes.
4) The final dosing formulation was a homogenous white cloudy suspension.

### Formulation Characterisation

Particle size distribution (PSD) of the suspension was determined as Dv₁₀=0.57 μm, Dv₅₀=2.41 μm and Dv₉₀=14.54 μm.

### Animal Dosing

Male Sprague Dawley (SD) rats with ~230-250 g body weight (ages 6-8 weeks) were used for the pharmacokinetic study. The animals had free access to food and water.

The Compound 1 suspension described above was administered via sub-cutaneous injection to each animal group (n=3) with a dosing volume of 3.33 mL/kg to achieve a 10 mg/kg dose.

At each time point, approximately 200 μL whole blood was collected in a K2EDTA tube via the jugular vein. Blood samples were put on ice and centrifuged at 2000 g for 5 min to obtain a plasma sample within 15 minutes. Plasma samples were stored at approximately −70° C. until analyzed by LC-MS.

### Results

The mean plasma concentration profile (n=3) after subcutaneous (SC) administration of the Compound suspension at a dose of 10 mg/kg is shown in FIG. 10. Prolonged exposure out to at least 58 days was achieved. Variability in plasma concentrations was low.

### Example 11: Compound 1 PK Study in Monkey

### Formulation Preparation

An aqueous suspension of Compound 1 free form (Form A) in a 0.9% NaCl+0.5% polysorbate 80 vehicle at a concentration of 3.0 mg/mL was prepared as follows:
1) Vehicle (0.9% w/w NaCl and 0.5% w/w polysorbate 80) was prepared by dissolving NaCl and polysorbate 80 in water.
2) Appropriate amount of Compound 1 was weighed and added to the vehicle.
3) The mixture was stirred for 15 minutes, then vortexed for 1 minute and then sonicated for 10 minutes.
4) The final dosing formulation was a homogenous white cloudy suspension.

### Animal Dosing

Male non-naïve Cyno Monkeys with ~4-6 kg body weight were used for the monkey PK study. The animals had free access to food and water. The prepared suspension was administered via subcutaneous injection to each animal group (n=3) with a dosing volume of 3.33 mL/kg to achieve a 10 mg/kg dose.

At each time point, approximately 0.5 mL whole blood was collected via the cephalic and saphenous vein into K2EDTA tubes. Blood samples were put on to ice and centrifuged at 2000 g for 5 min to obtain plasma sample within 15 minutes. Plasma samples were stored at approximately −70° C. until analyzed by LC-MS.

### Results

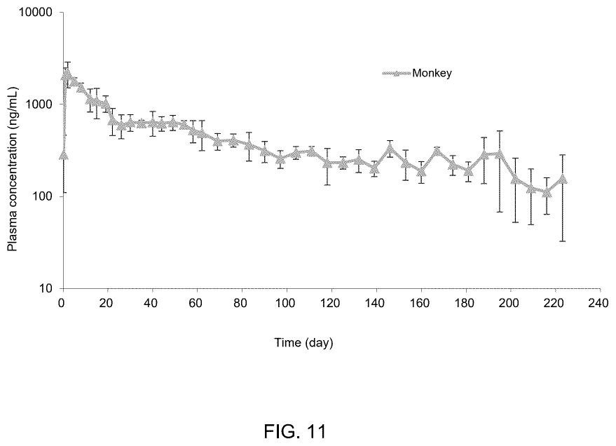

The mean plasma concentration profile (n=3) after subcutaneous (SC) administration of the Compound 1 suspension is shown in FIG. 11. Remarkably, following an initial drug burst, prolonged exposure was achieved out to at least 220 days at the administered dosing level of 10 mg/kg.

### Example 12: Compound 1 PK Study in Dog

### Formulation Preparation

An aqueous suspension of Compound 1 free form (Form A) in a 0.9% NaCl+0.5% poloxamer 188 vehicle at a concentration of 5.0 mg/mL was prepared as follows:
1) Vehicle (0.9% w/w NaCl and 0.5% w/w poloxamer 188) was prepared by dissolving NaCl and poloxamer 188 in water.
2) Appropriate amount of Compound 1 free form was weighed and added to the vehicle.
3) The mixture was stirred for 15 minutes, then vortexed for 1 minute and then sonicated for 10 minutes.
4) The final dosing formulation was a homogenous white cloudy suspension.

### Animal Dosing

Male non-naïve beagles with ~10 kg body weight were used for the dog PK study. The animals had free access to food and water. The prepared suspension was administered via subcutaneous injection to each animal group (n=3) with a dosing volume of 2 mL/kg to achieve a 10 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Results

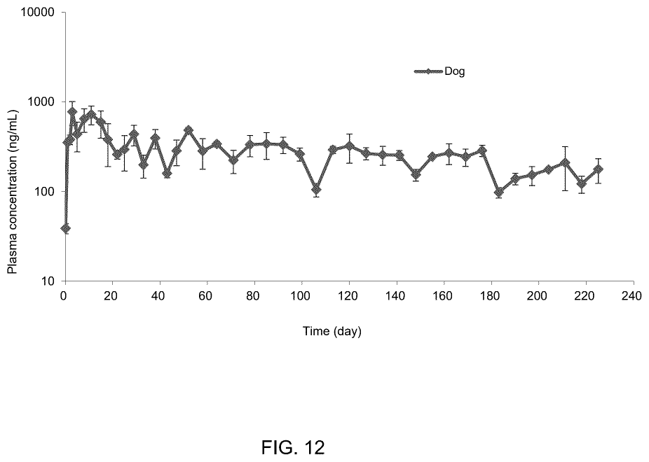

The mean plasma concentration profile (n=3) after subcutaneous (SC) administration of the Compound 1 suspension is shown in FIG. 12. Following an initial drug burst, prolonged exposure was achieved out to at least 220 days at the administered dosing level of 10 mg/kg.

### Summary from Example 10-12 Compound 1 PK Studies

In 3 different species, injectable long acting depot formulations containing Compound 1 have been found to provide continuous and steady plasma levels of the drug for significantly prolonged periods of time following a single subcutaneous administration.

In addition, there were no adverse reactions observed at the injection sites in the study animals.

### Example 13: PK Studies in Rat, Monkey and Dog Following IV Administration

### Formulation Preparation—Rat

A 0.2 mg/mL solution of Compound 1 in 10% NMP, 10% Solutol HS15 and 80% Saline was prepared as follows:
1) A stock solution of Compound 1 in NMP was prepared.
2) An equal volume of the Compound 1 stock solution and Solutol HS15 were combined and mixed.
3) The above solution was diluted with normal saline to reach the target concentration. The final formulation was a clear solution.

### Animal Dosing—Rat

Male Sprague Dawley (SD) rats with 230-250 g body weight, ages 6-8 weeks, were used for the rat pharmacokinetic study. The animals had free access to food and water. The Compound 1 solution described above was administered via intravenous injection to animals (n=3) to achieve a 1.0 mg/kg dose.

At each time point, approximately 200 μL whole blood was collected in a K2EDTA tubes. Blood samples were put on ice and centrifuged at 2000 g for 5 min to obtain a plasma sample within 15 minutes. Plasma samples were stored at approximately −70° C. until analyzed by LC-MS according to the method described above.

### Results

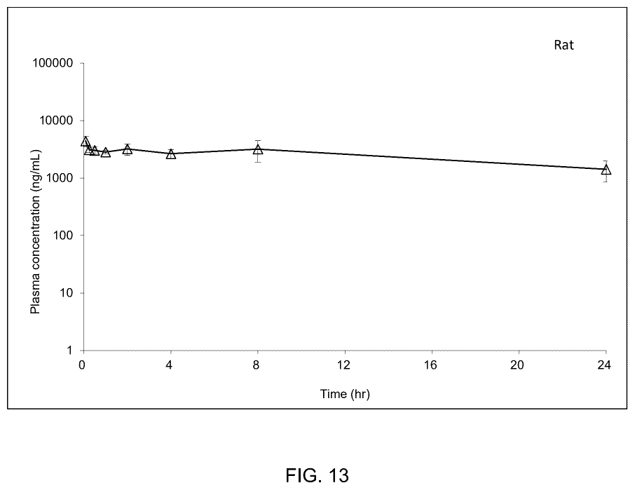

The mean plasma concentration profiles (n=3) after intravenous (IV) to rat at 1.0 mg/kg of Compound 1 solution can be found in FIG. 13.

### Formulation Preparation—Monkey

A 0.25 mg/mL solution of Compound 1 in 10% DMSO, 10% Solutol HS15 and 80% Saline was prepared as follows:
1) A stock solution of Compound 1 in DMSO was prepared.
2) An equal volume of the Compound 1 stock solution and Solutol HS15 were combined and mixed.
3) The above solution was diluted with normal saline to reach the target concentration. The final formulation was a clear solution.

### Animal Dosing—Monkey

Male non-naïve Cyno Monkeys with were used for the monkey PK study. The animals had free access to food and water. The prepared solution was administered via intravenous injection to each animal group (n=3) to achieve a 0.25 mg/kg dose.

At each time point, approximately 0.5 mL whole blood was collected into K2EDTA tubes. Blood samples were put on to ice and centrifuged at 2000 g for 5 min to obtain plasma sample within 15 minutes. Plasma samples were stored at approximately −70° C. until analyzed by LC-MS.

### Results

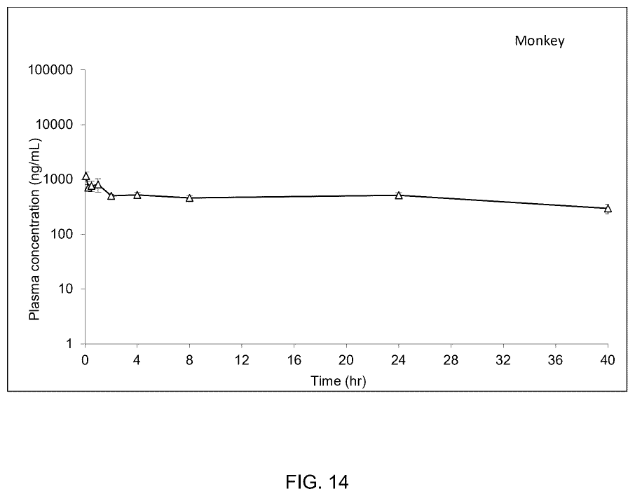

The mean plasma concentration profiles (n=3) after intravenous (IV) at 0.25 mg/kg of Compound 1 solution can be found in FIG. 14.

### Formulation Preparation—Dog

A 0.15 mg/mL solution of Compound 1 in 10% DMSO, 10% Solutol HS15 and 80% Saline was prepared as follows:
1) A stock solution of Compound 1 in DMSO was prepared.
2) An equal volume of the Compound 1 stock solution and Solutol HS15 were combined and mixed.
3) The above solution was diluted with normal saline to reach the target concentration. The final formulation was a clear solution.

### Animal Dosing—Dog

Male non-naïve beagles were used for the dog PK study. The animals had free access to food and water. The prepared solution was administered via subcutaneous injection to each animal group (n=3) to achieve a 0.15 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Results

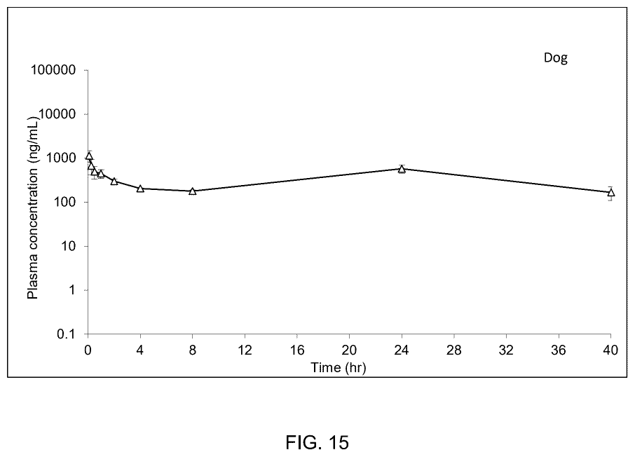

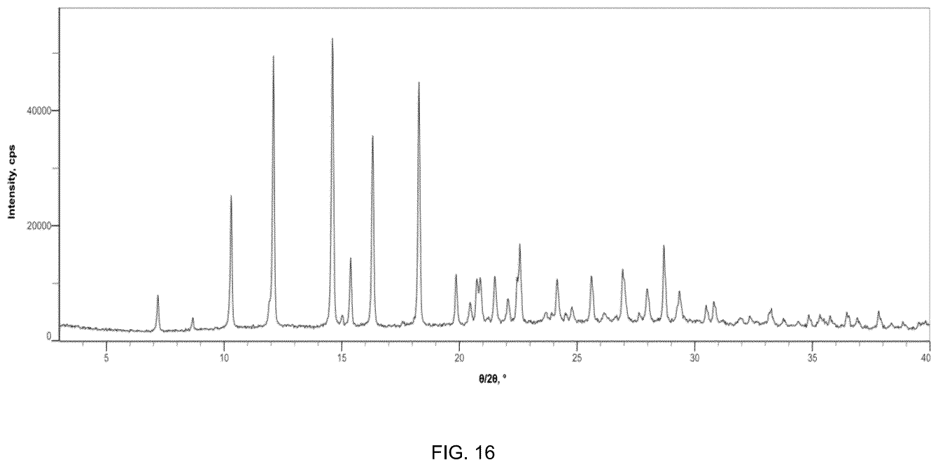

The mean plasma concentration profiles (n=3) after intravenous (IV) at 0.15 mg/kg of Compound 1 solution can be found in FIG. 15.

### Summary

IV dosing of Compound 1 in rat, monkey and dog at doses of 1 mg/kg, 0.25 mg/kg and 0.15 mg/kg respectively gave the long terminal half-lives as shown in the following table:

| | Compound 1 |
|---|---|
| Rat t_{1/2} (hr) | 22 |
| Monkey t_{1/2} (hr) | 49.6 |
| Dog t_{1/2} (hr) | 34.9 |

### Example 13A: Further Compound 1 PK Studies in Rat, Monkey, Dog and Mini-Pig Following IV Administration

### Formulation Preparation—Rat

A 0.2 mg/mL solution of Compound 1 in 10% NMP, 10% Solutol HS15 and 80% Saline was prepared as follows:
1) A stock solution of Compound 1 in NMP was prepared.
2) An equal volume of the Compound 1 stock solution and Solutol HS15 were combined and mixed.
3) The above solution was diluted with normal saline to reach the target concentration. The final formulation was a clear solution.

### Animal Dosing—Rat

Male Sprague Dawley (SD) rats with 180-184 g body weight, ages 6-8 weeks, were used for the rat pharmacokinetic study. The animals had free access to food and water. The Compound 1 solution described above was administered via intravenous injection (1 mL/kg) to each animal (n=3) to achieve a 0.2 mg/kg dose.

At each time point, approximately 200 μL whole blood was collected in a K2EDTA tubes. Blood samples were put on ice and centrifuged at 2000 g for 5 min to obtain a plasma sample within 15 minutes. Plasma samples were stored at approximately −70° C. until analyzed by LC-MS according to the method described above.

### Formulation Preparation—Monkey

A 0.2 mg/mL solution of Compound 1 in 10% DMSO, 10% Solutol HS15 and 80% Saline was prepared as follows:
1) A stock solution of Compound 1 in DMSO was prepared.
2) An equal volume of the Compound 1 stock solution and Solutol HS15 were combined and mixed.
3) The above solution was diluted with normal saline to reach the target concentration. The final formulation was a clear solution.

### Animal Dosing—Monkey

Male non-naïve Cyno Monkeys with were used for the monkey PK study. The animals had free access to food and water. The Compound 1 solution described above was administered via intravenous injection (1 mL/kg) to each animal (n=3) to achieve a 0.2 mg/kg dose.

At each time point, approximately 0.5 mL whole blood was collected into K2EDTA tubes. Blood samples were put on to ice and centrifuged at 2000 g for 5 min to obtain plasma sample within 15 minutes. Plasma samples were stored at approximately −70° C. until analyzed by LC-MS.

### Formulation Preparation—Dog

A 0.15 mg/mL solution of Compound 1 in 10% DMSO, 10% Solutol HS15 and 80% Saline was prepared as follows:
1) A stock solution of Compound 1 in DMSO was prepared.
2) An equal volume of the Compound 1 stock solution and Solutol HS15 were combined and mixed.
3) The above solution was diluted with normal saline to reach the target concentration. The final formulation was a clear solution.

### Animal Dosing—Dog

Male non-naïve beagles were used for the dog PK study. The animals had free access to food and water. The Compound 1 solution described above was administered via intravenous injection (1 mL/kg) to each animal (n=3) to achieve a 0.15 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Formulation Preparation—Minipig

A 0.25 mg/mL solution of Compound 1 in 10% DMSO, 10% Solutol HS15 and 80% Saline was prepared as follows:
4) A stock solution of Compound 1 in DMSO was prepared.
5) An equal volume of the Compound 1 stock solution and Solutol HS15 were combined and mixed.
6) The above solution was diluted with normal saline to reach the target concentration. The final formulation was a clear solution.

### Animal Dosing—Minipig

Naïve Bama Pigs (15-16 kg) were used for the minipig PK study. The animals had free access to food and water. The Compound 1 solution described above was administered via intravenous injection (1 mL/kg) to each animal (n=3) to achieve a 0.25 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Results

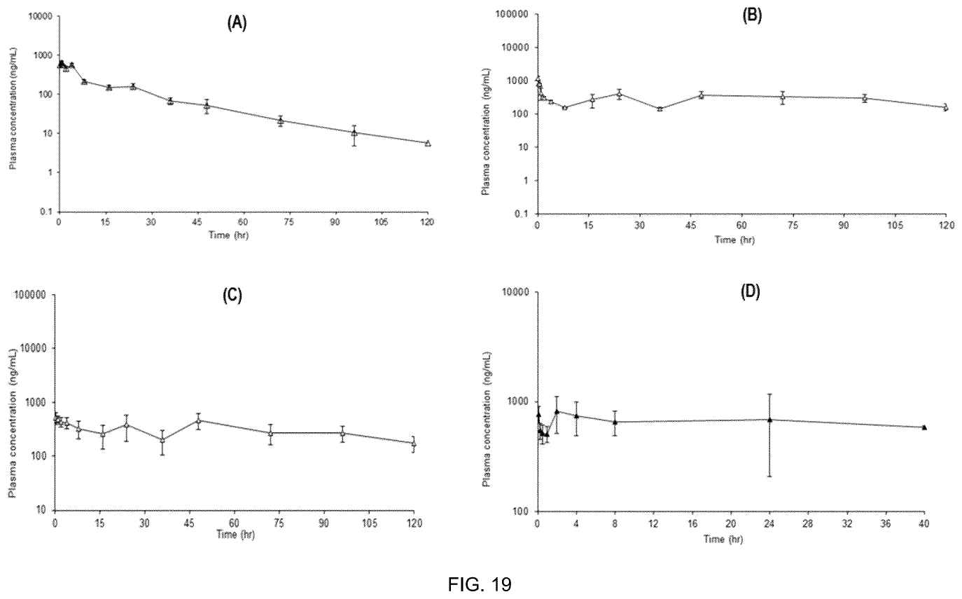

The mean plasma concentration profiles (n=3) after intravenous (IV) injection of Compound 1 solution in rat, dog, monkey and mini-pig can be found in FIG. 19 (A)-(D).

### Summary

IV dosing of Compound 1 in rat, dog, monkey and mini-pig at doses of 0.2 mg/kg, 0.15 mg/kg, 0.2 mg/kg and 0.25 mg/kg respectively gave the terminal half-lives and clearances as shown in the following table:

| Species | Half-life (days) | Clearance (L/hr/kg) |
|---|---|---|
| Rat | 0.8 | 0.02 |
| Dog | 2.3 | 0.002 |
| Monkey | 3.0 | 0.004 |
| Mini-pig | 5.6 | 0.0018 |

Based on the above PK data in multiple species, and using allometric scaling, the predicted human biological terminal half-life of Compound 1 is 7.6 days (182 hours) with a clearance of 0.06 L/hr.

### Example 14: Pritelivir Sub-Cutaneous PK Study in Dog

### (A) Formulation Preparation

### Solution Formulation:

A solution of pritelivir in 90% NMP (Sigma) and 10% Solutol HS15 (BASF) at a concentration of 100 mg/mL was prepared as follows:
1) Around 460 mg of crystalline pritelivir free base sesquihydrate (based on assay) was weighed and added to ~3.9 mL of NMP. The mixture was stirred for 10 mins.
2) After the pritelivir had dissolved, Solutol HS15 (−0.4 mL) was added to the solution and mixed for another 2 mins.
3) The final dosing formulation was a clear solution.

### Aqueous Micro-Suspension:

An aqueous micro-suspension of crystalline pritelivir free base (sesquihydrate) in a 2 mg/mL polysorbate 80 (Tween 80) and 5 mg/mL Methocel A4M vehicle at a concentration of 100 mg/mL was prepared as follows:
1) Crystalline pritelivir free base (sesquihydrate) was jet milled [grinding pressure 0.8 mPa] to obtain microparticles (Dv90<10 um).
2) Around 460 mg of jet milled material (based on assay) were then dispersed in a 2 mg/mL polysorbate 80 (Tween 80) and 5 mg/ml Methocel A4M solution (−4.4 mL) to obtain a 100 mg/ml suspension.
3) The mixture was stirred for 10 minutes.
4) The final dosing formulation was a homogenous white cloudy suspension.

### Formulation Characterisation of Micro-Suspension:

Particle size distribution (PSD) of the micro-suspension was determined as Dv₁₀=1.96 μm, Dv₅₀=4.17 μm and Dv₉₀=8.51 μm.

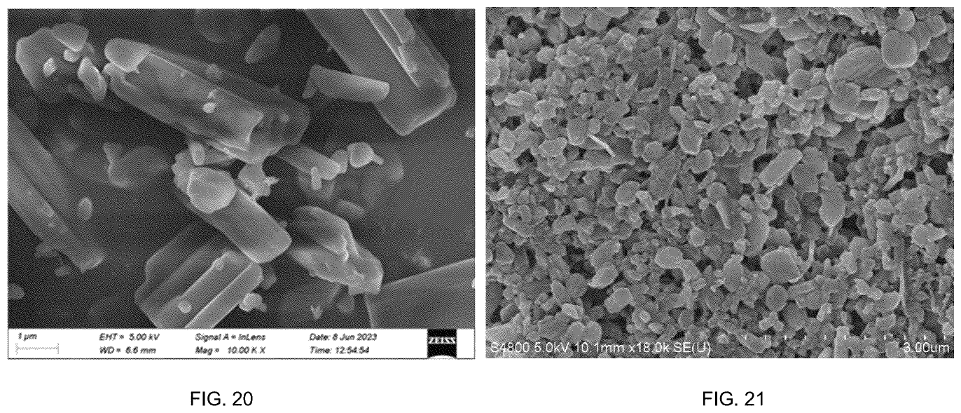

An SEM image of the micro-suspension can be found in FIG. 20.

### Aqueous Nano-Suspension:

An aqueous nano-suspension of crystalline pritelivir free base (sesquihydrate) in a 10 mg/mL NaCMC, 2 mg/mL polysorbate 80 (Tween 80) and 50 mg/mL mannitol vehicle at a concentration of 100 mg/mL was prepared as follows:
1) 10 mg/mL crystalline pritelivir free base (sesquihydrate) was dispersed into a solution of 1 mg/mL NaCMC+0.2 mg/mL polysorbate 80 (Tween 80) and 5 mg/mL mannitol.
2) The suspension was subjected to high pressure homogenizer (around 20 cycles at 600 bar followed with 130 cycles at 900 bar) until particle size was around 1 μm.
3) After particle size reduction, the suspension was subjected to freeze drying to obtain a freeze-dried powder.
4) The freeze-dried powder was reconstituted with water to obtain a 100 mg/ml suspension.
5) The suspension was wet milled (10:1 0.1 mm zirconium oxide milling beads: pritelivir) at 700 rpm for 500 minutes to reduce particle size to nanosized particles (Dv90<500 nm).
6) This nanosuspension was subjected to freeze drying to obtain a freeze-dried powder.
7) Appropriate amounts of freeze-dried powder (based on assay) were weighed and added to water to obtain a 100 mg/ml suspension.
8) The mixture was stirred for 10 minutes.
9) The final dosing formulation was a homogenous white cloudy suspension.

An SEM image of the nano-suspension can be found in FIG. 21.

### (B) Animal Dosing

Male non-naïve beagles were used for the dog PK study. The animals had free access to food and water. The prepared formulations were administered via subcutaneous injection to each animal group (n=3) with a dosing volume of 0.1 mL/kg to achieve a 10 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Results

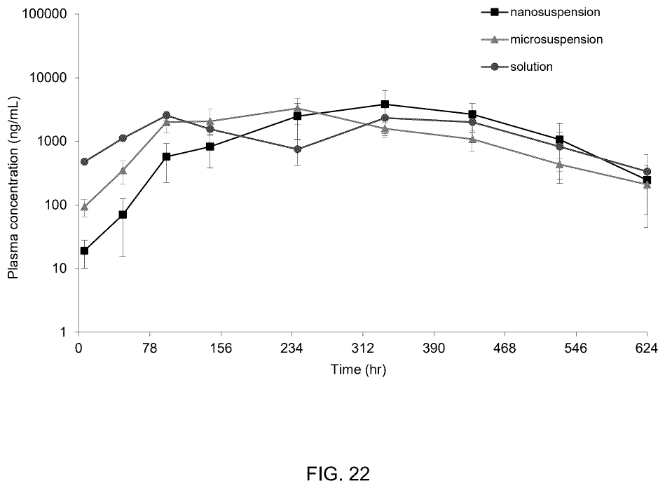

The mean plasma concentration profile (n=3) after subcutaneous (SC) administration of the pritelivir formulations is shown in FIG. 22. Prolonged exposure was achieved at the administered dosing level using all three formulations. The solution exhibited the highest plasma concentration levels at the early time points. Surprisingly, for the aqueous suspension formulations at the early time points (up to 234 hr), higher plasma concentrations of pritelivir were obtained from the microsuspension, rather than from the nanosuspension formulation. The formulations were able to achieve prolonged release using a high concentration of pritelivir and dosing could therefore be carried out using a small dosing volume of just 0.1 mL/kg. In addition, no adverse reactions were observed at the injection sites of the study animals.

### Example 15: Further Compound 1 PK Studies in Rat

### Formulation Preparation

A solution of Compound 1 in 90% NMP and 10% Solutol HS15 at a concentration of 50 mg/mL was prepared as follows:
1) An appropriate amount of crystalline Compound 1 (Form C) was weighed and added to NMP.
2) After Compound 1 had dissolved, Solutol HS15 was added to the solution and mixed [state mixing conditions and time].
3) The final dosing formulation was a clear solution.

### Animal Dosing

Male Sprague Dawley (SD) rats with ~230-250 g body weight (aged 6-8 weeks) were used for the pharmacokinetic study. The animals had free access to food and water. The Compound 1 solution described above was administered via sub-cutaneous injection to each animal group (n=3) with a dosing volume of 2 mL/kg to achieve a 100 mg/kg dose.

At each time point, approximately 150 μL whole blood was collected in a K2EDTA tube via the jugular vein. Blood samples were put on ice and centrifuged at 2000 g for 5 min to obtain a plasma sample within 15 minutes. Plasma samples were stored at approximately −70° C. until analyzed by LC-MS.

### Results

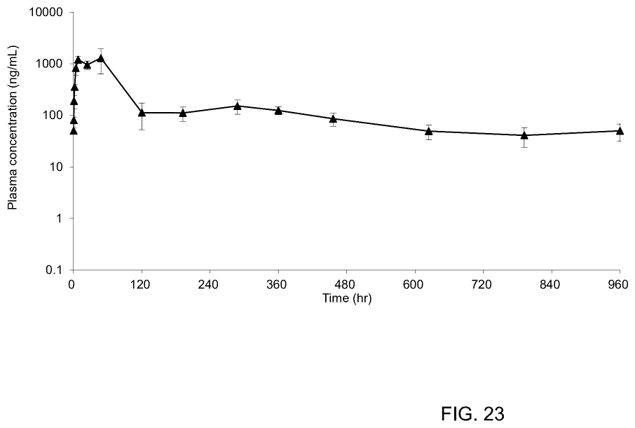

The mean plasma concentration profile (n=3) after subcutaneous administration of the Compound 1 solution at a dose of 50 mg/kg is shown in FIG. 23. Following an initial drug burst, prolonged exposure was achieved at the administered dosing level for 960 hours (length of the study). Variability in plasma concentrations was low. In addition, no adverse reactions were observed at the injection sites of the study animals.

### Example 16: Further Compound 1 PK Studies in Dog

### Formulation Preparation

A solution of Compound 1 in 90% NMP and 10% Solutol HS15 at a concentration of 50 mg/mL was prepared as follows:
1) An appropriate amount of crystalline Compound 1 (Form C) was weighed and added to NMP.
2) After Compound 1 had dissolved, Solutol HS15 was added to the solution and mixed.
3) The final dosing formulation was a clear solution.

A solution of Compound 1 in 90% NMP and 10% Solutol HS15 at a concentration of 100 mg/mL was prepared as follows:
1) An appropriate amount of crystalline Compound 1 (Form C) was weighed and added to NMP.
2) After Compound 1 had dissolved, Solutol HS15 was added to the solution and mixed.
3) The final dosing formulation was a clear solution.

### Animal Dosing

Male non-naïve beagles were used for the dog PK study. The animals had free access to food and water. The prepared 50 mg/mL solution was administered via subcutaneous injection to each animal group (n=3) with a dosing volume of 0.2 mL/kg to achieve a 10 mg/kg dose. The prepared 100 mg/mL solution was administered via subcutaneous injection to each animal group (n=3) with a dosing volume of 0.1 mL/kg to achieve a 10 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Results

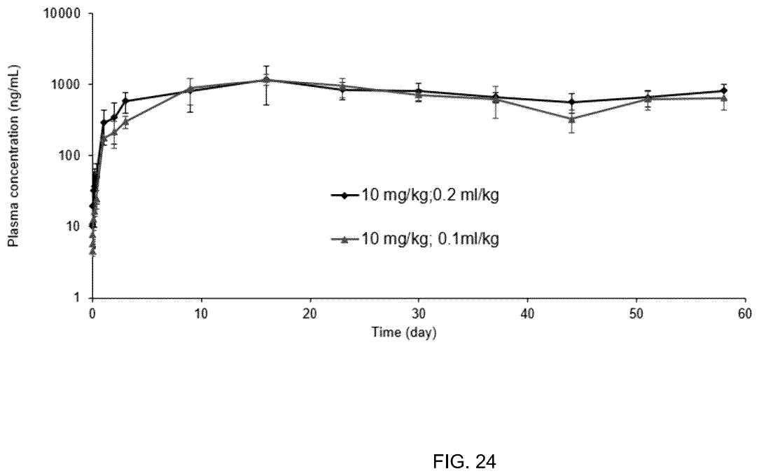

The mean plasma concentration profile (n=3) after subcutaneous administration of the Compound 1 solution at the two dosing volumes is shown in FIG. 24. Prolonged exposure was achieved for at least 60 days (length of the study) at the administered dosing level using both formulations. The formulations were able to achieve prolonged release using small dosing volumes. In addition, no adverse reactions were observed at the injection sites of the study animals

### Example 17: Compound 1 Sub-Cutaneous and Intra-Muscular Administration PK Study in Rat Using Microsuspension and Nanosuspension Formulations

### (A) Formulation Preparation

### Aqueous Microsuspension:

An aqueous microsuspension of crystalline Compound 1 free form (crystalline Form C) in a 20 mg/mL PVP K30, 10 mg/mL poloxamer 188 and 50 mg/mL mannitol vehicle at a concentration of 200 mg/mL was prepared as follows:
1) Crystalline Compound 1 (Form C) was jet milled to obtain microparticles (Dv90<10 μm).
2) Jet milled material was dispersed in 20 mg/ml PVP K30+10 mg/ml Poloxamer 188+50 mg/ml mannitol to obtain a homogeneous suspension.
3) The suspension was subjected to freeze drying to obtain a freeze-dried powder.
4) Appropriate amounts of freeze-dried powder (based on assay) were weighed and added to water to obtain a 200 mg/ml suspension.
5) The mixture was stirred for 5 minutes.
6) The final dosing formulation was a homogenous white cloudy suspension.

### Formulation Characterisation of Micro-Suspension:

Particle size distribution (PSD) of the micro-suspension was determined as Dv₁₀=1.19 μm, Dv₅₀=2.76 μm and Dv₉₀=5.65 μm.

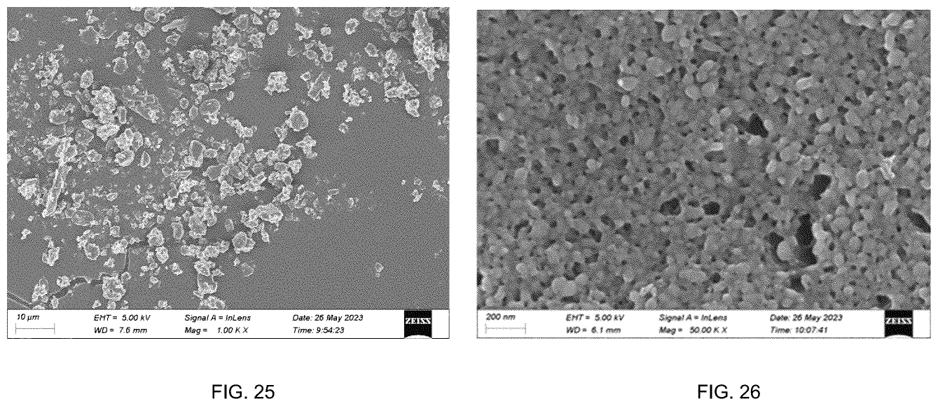

An SEM image of the microsuspension can be found in FIG. 25.

### Aqueous Nano-Suspension:

An aqueous nano-suspension of crystalline Compound 1 free form (crystalline Form C) in a 20 mg/mL PVP K30, 10 mg/mL poloxamer 188 and 50 mg/mL mannitol vehicle at a concentration of 200 mg/mL was prepared as follows:
1) 20 mg/mL crystalline Compound 1 (Form C) was dispersed into a solution of 2 mg/mL PVP K30 1.0 mg/mL poloxamer 188 and 5 mg/mL mannitol
2) The suspension was subjected to high pressure homogenizer until the size was around 1 μm.
3) After particle size reduction, the suspension was subjected to freeze drying to obtain a freeze-dried powder.
4) The freeze-dried powder was reconstituted with water to obtain a 100 mg/mL suspension.
5) The suspension was wet milled (10:1 0.1 mm zirconium oxide milling beads: Compound 1) at 900 rpm for 600 minutes to reduce particle size to nanosized particles (Dv90<200 nm).
6) This nanosuspension was subjected to freeze drying to obtain a freeze-dried powder.
7) Appropriate amounts of freeze-dried powder (based on assay) were weighed and added to water to obtain a 200 mg/ml suspension.
8) The mixture was stirred for 10 minutes.
9) The final dosing formulation was a homogenous white cloudy suspension.

### Formulation Characterisation of Nano-Suspension:

Particle size distribution (PSD) of the nano-suspension was determined as Dv₁₀=69.1 nm, Dv50=77.7 nm, and Dv90=88.5 nm.

An SEM image of the nano-suspension can be found in FIG. 26.

### (B) Animal Dosing

Male Sprague Dawley (SD) rats with ~230-250 g body weight (aged 6-8 weeks) were used for the pharmacokinetic study. The animals had free access to food and water. The Compound 1 solution described above was administered via sub-cutaneous or intra-muscular injection to each animal group (n=3) with a dosing volume of 0.1 mL/animal (~0.4 mL/kg) to achieve a 20 mg/animal dose (~80 mg/kg).

At each time point, approximately 150 μL whole blood was collected in a K2EDTA tube via the jugular vein. Blood samples were put on ice and centrifuged at 2000 g for 5 min to obtain a plasma sample within 15 minutes. Plasma samples were stored at approximately −70° C. until analyzed by LC-MS.

### Results

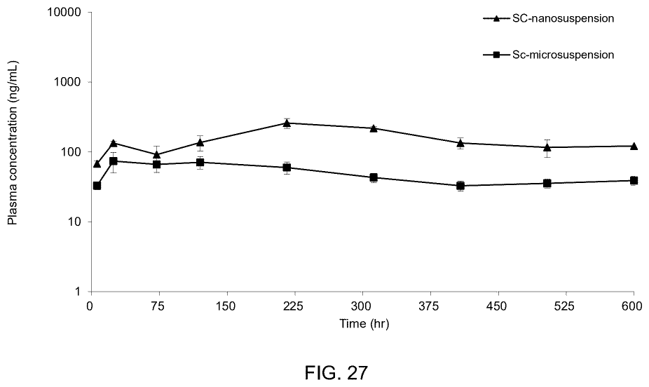

The mean plasma concentration profiles (n=3) after subcutaneous administration of the Compound 1 micro- and nano-suspensions can be found in FIG. 27. Both formulations were able to provide prolonged exposure for at least 600 hours (length of the study). The nanosuspension provided higher plasma levels than the microsuspension throughout the test period. The formulations were able to achieve prolonged release using high drug loading and small dosing volumes. In addition, there were no adverse reactions observed at the injection sites in the study animals.

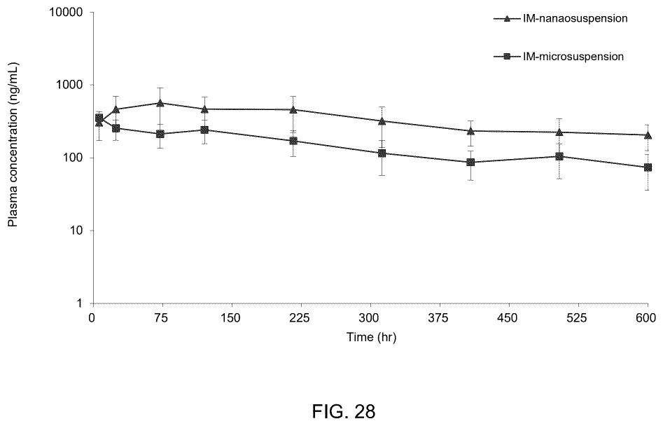

The mean plasma concentration profiles (n=3) after intramuscular administration of the Compound 1 micro- and nano-suspensions can be found in FIG. 28. Both formulations were able to provide prolonged exposure for at least 600 hours (length of the study) and plasma levels were higher than those achieved by these formulations when administered by subcutaneous injection. The nanosuspension provided higher plasma levels than the microsuspension. The formulations were able to achieve prolonged release using high drug loading and small dosing volumes. In addition, there were no adverse reactions observed at the injection sites in the study animals.

1. A long-acting injectable depot pharmaceutical composition comprising:
(a) a helicase-primase inhibitor or a pharmaceutically acceptable salt thereof; and
(b) one or more pharmaceutically acceptable excipients;
wherein the helicase-primase inhibitor has a human biological terminal half-life of 10 hours or more.

2. The composition according to claim 1, wherein the composition is a subcutaneous or intramuscular long-acting injectable depot pharmaceutical composition.

3. The composition according to claim 2, wherein the composition is for administration twice a month, once a month, once every two months, once every three months, once every six months or once every year.

4. The composition according to claim 1, wherein the helicase-primase inhibitor has an in-vitro EC₅₀ value of less than 0.1 μM against HSV-1 and/or HSV-2.

5. The composition according to claim 1, wherein the helicase-primase inhibitor is N-Methyl-N-(4-methyl-5-sulfamoyl-1,3-thiazol-2-yl)-2-[4-(pyridin-2-yl)phenyl]acetamide: 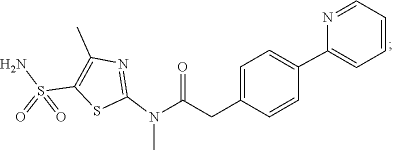 [SMILES: Cc1nc(N(C)C(=O)Cc2ccc(-c3ccccn3)cc2)sc1S(N)(=O)=O]
or a pharmaceutically acceptable salt thereof.

6. The composition according to claim 1, wherein the helicase-primase inhibitor is N-Methyl-N-(4-methyl-5-sulfamoyl-1,3-thiazol-2-yl)-2-[4-(pyridin-2-yl)phenyl]acetamide free base.

7. The composition according to claim 6, wherein the free base is present as a crystalline solid.

8. The composition according to claim 6, wherein the long-acting injectable depot pharmaceutical composition is an aqueous suspension and the free base is suspended as a crystalline solid.

9. The composition according to claim 8, wherein the long-acting injectable depot pharmaceutical composition is an aqueous nanosuspension and the free base is suspended as a crystalline solid.

10. The composition according to claim 1, wherein the long-acting injectable depot pharmaceutical composition comprises a solution in organic solvent based formulation.

11. The composition according to claim 10, wherein the organic solvent is selected from benzyl alcohol, benzyl benzoate, N-methyl-pyrrolidone (NMP), 2-pyrrolidone, ethanol, propylene glycol, acetone, methyl acetate, ethyl acetate, methyl ethyl ketone, dimethylacetamide (DMA), dimethylformamide, dimethyl sulfoxide (DMSO), tetrahydrofuran, caprolactam, oleic acid, 1-dodecylazacycloheptan-2-one, and mixture thereof.

12. The composition according to claim 10, wherein the helicase-primase inhibitor is N-Methyl-N-(4-methyl-5-sulfamoyl-1,3-thiazol-2-yl)-2-[4-(pyridin-2-yl)phenyl]-acetamide and the organic solvent is selected from NMP, DMA and DMSO.

13. The composition according to claim 1, wherein the helicase-primase inhibitor is 2-(3-(2′,5′-difluoro-[1,1′-biphenyl]-4-yl)-2-oxotetrahydropyrimidin-1(2H)-yl)-4-methyl-thiazole-5-sulfonamide (Compound 1): 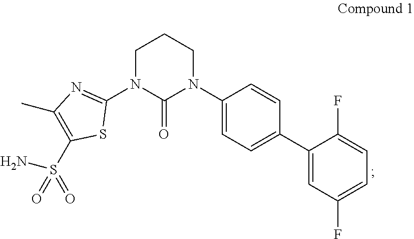 [SMILES: Cc1nc(N2CCCN(c3ccc(-c4cc(F)ccc4F)cc3)C2=O)sc1S(N)(=O)=O]
or a pharmaceutically acceptable salt thereof.

14. The composition according to claim 1, wherein the helicase-primase inhibitor is 2-(3-(2′,5′-difluoro-[1,1′-biphenyl]-4-yl)-2-oxotetrahydropyrimidin-1(2H)-yl)-4-methylthiazole-5-sulfonamide free form.

15. The composition according to claim 14, wherein the free form is present as a crystalline solid.

16. The composition according to claim 14, wherein the long-acting injectable depot pharmaceutical composition is an aqueous suspension and the free form is suspended as a crystalline solid.

17. The composition according to claim 16, wherein the long-acting injectable depot pharmaceutical composition is an aqueous microsuspension or nanosuspension and the free form is suspended as a crystalline solid.

18. The composition according to claim 13, wherein the long-acting injectable depot pharmaceutical composition comprises a solution in organic solvent based formulation.

19. The composition according to claim 18, wherein the organic solvent is selected from N-methyl pyrrolidone (NMP), dimethylacetamide (DMA), dimethyl sulfoxide (DMSO), benzyl alcohol, benzyl benzoate and mixtures thereof.

20. The composition according to claim 19, wherein the organic solvent is selected from NMP, DMA and DMSO.

21. The composition according to claim 1, wherein the long-acting injectable depot pharmaceutical composition comprises about 2 wt % to about 40 wt % of a helicase-primase inhibitor or a pharmaceutically acceptable salt thereof.

22. The composition according to claim 1, wherein the long-acting injectable depot pharmaceutical composition comprises about 100 mg to about 1200 mg, about 10 mg to about 900 mg or about 100 mg to about 600 mg of a helicase-primase inhibitor or a pharmaceutically acceptable salt thereof.

23. The composition according to claim 1, wherein the long-acting injectable depot pharmaceutical composition comprises N-Methyl-N-(4-methyl-5-sulfamoyl-1,3-thiazol-2-yl)-2-[4-(pyridin-2-yl)phenyl]acetamide free base as the helicase-primase inhibitor and the composition produces a plasma concentration of N-Methyl-N-(4-methyl-5-sulfamoyl-1,3-thiazol-2-yl)-2-[4-(pyridin-2-yl)phenyl]acetamide in the subject after administration of at least 25 ng/mL for at least 80% of the dosing interval, wherein the dosing interval is at least 10 days.

24. The composition according to claim 1, wherein the long-acting injectable depot pharmaceutical composition comprises 2-(3-(2′,5′-difluoro-[1,1′-biphenyl]-4-yl)-2-oxotetrahydropyrimidin-1 (2H)-yl)-4-methylthiazole-5-sulfonamide free form as the helicase-primase inhibitor and the composition produces a plasma concentration of 2-(3-(2′,5′-difluoro-[1,1′-biphenyl]-4-yl)-2-oxotetrahydropyrimidin-1(2H)-yl)-4-methylthiazole-5-sulfonamide in the subject after administration of at least 25 ng/mL for at least 80% of the dosing interval, wherein the dosing interval is at least 10 days.

25. The composition according to claim 1, wherein the composition is stable for at least four weeks.

26. (canceled)

27. A method for treating a herpes virus (such as HSV) infection in a subject in need thereof, the method comprising administering a therapeutically effective amount of a helicase-primase inhibitor, or a pharmaceutically acceptable salt thereof, to the subject wherein the helicase-primase inhibitor or a pharmaceutically acceptable salt thereof, is formulated as a pharmaceutical composition according to claim 1.

28. The method according to claim 27, wherein the pharmaceutical composition is administered twice a month, once a month, once every two months, once every three months, once every six months, or once every year.

29. The method according to claim 27, wherein the pharmaceutical composition is administered once a month, or once every three months.
